(19) United States
(12) Patent Application Publication
(10) Pub. No.: US 2021/0383899 A1
(43) Pub. Date: Dec. 9, 2021

(54) Medical Screening System and Method

(71) Applicant: Prime Medical, LLC, Baton Rouge, LA (US)

(72) Inventors: Sean H. Connor, Baton Rouge, LA (US); Luke P. Lee, Baton Rouge, LA (US)

(21) Appl. No.: 16/892,090

(22) Filed: Jun. 3, 2020

(51) Int. Cl. G16H 10/20; G16H 50/20; G16H 40/20; G06Q 10/10; G16H 70/20; G16H 10/60; G06Q 50/26

(52) U.S. Cl. CPC G16H 10/20; G16H 50/20; G16H 40/20; G06F 3/048; G16H 70/20; G16H 10/60; G06Q 50/265; G06Q 10/105

(57) ABSTRACT

A medical screening system and method for screening potential entrants to a facility for medical conditions prior to entry. The medical screening system and method generally includes an evaluation system and an interface. The interface resides on a portable electronic device and the evaluation system may reside on the same device or comprise a remote centralized system. The evaluation system automatically selects an appropriate questionnaire eliciting information regarding the medical condition of a potential entrant from a plurality of questionnaires and provides it to the interface for display. The potential entrant provides responses to the questionnaire, and the evaluation system automatically evaluates the responses, generates an entry decision, and sends an indication to the interface. The interface provides an entry decision presentation comprising one or more perceptible elements and entry to the facility by the potential entrant is controlled based on the entry decision presentation.

### CROSS REFERENCE TO RELATED APPLICATIONS

Not applicable to this application.

### STATEMENT REGARDING FEDERALLY SPONSORED RESEARCH OR DEVELOPMENT

Not applicable to this application.

### BACKGROUND

### Field

Example embodiments in general relate to a medical screening system and method for screening persons for disease, infection, or another condition prior to entry to a facility.

### Related Art

Any discussion of the related art throughout the specification should in no way be considered as an admission that such related art is widely known or forms part of common general knowledge in the field.

It is widely recognized that persons who have been exposed to an infectious disease or condition and/or who have the symptoms of an infectious disease or condition should avoid contact with other persons and with places where other persons may be present in order to avoid exposing and possibly infecting them. One way for exposed and/or symptomatic persons to avoid such contact is to self-assess their exposure and symptoms, and to self-quarantine. Several on-line questionnaires designed to assist persons in self-screening and self-reporting may exist.

Proprietors of facilities and premises, for example employers, also have an interest in knowing whether persons seeking access to their facilities and premises have been exposed to an infectious disease or condition and/or may be symptomatic. In particular, employers and other proprietors have an interest in knowing such information before a person is allowed to enter their facilities and premises. If such information could be made available to employers and other proprietors before persons enter their facilities and premises, they could make informed decisions and take appropriate actions regarding persons who should and should not be permitted entry in order to prevent exposure and possible infection to themselves, their employees, customers, and others.

Existing on-line self-screening and self-reporting questionnaires do not provide information to the employers and other proprietors of facilities and premises to enable them to make decisions and to take appropriate actions to control entry. Even if employers and other proprietors had access to such information prior to entry, it would be an overwhelming task for them to manually evaluate the information and make accurate entry decisions in real time, especially in high volume entry situations such as restaurants, stadiums, etc.

There is a need for a medical screening system and method that can obtain information from persons seeking entry to facilities or premises regarding their exposure to an infectious disease or condition and/or symptoms prior to such persons entering the facilities or premises. There is a need for such a medical screening system and method that can obtain such information without burdening the employers or other proprietors of such facilities or premises. There is a need for such a medical screening system and method that can automatically evaluate such information, automatically generate entry decisions based on such information, and automatically report such entry decisions to employers and other proprietors to enable them to control entry to their facilities and premises substantially in real time and without substantial burden to the employers and other proprietors. There is a need for such a medical screening system and method that enables employers and other proprietors to respond to decisions denying entry to persons direct such persons to appropriate healthcare facilities for appropriate evaluation.

### SUMMARY

An example embodiment is directed to a medical screening system and method. The example embodiment of the medical screening system includes an electronic medical evaluation system and a potential entrant interface. The electronic medical evaluation system comprises at least one questionnaire including one or more questions concerning a medical condition or symptom of a potential entrant to a facility. The questions may relate to one or more of potential entrant identification, potential entrant type, potential entrant job type, potential entrant job identification, potential entrant entry purposes, travel locations, exposure to disease, symptoms, tests performed, test results, and diagnoses received.

The potential entrant interface is configured to receive the questionnaire from the electronic medical evaluation system and to display the questionnaire for a potential entrant to provide responses to the questions. The electronic medical evaluation system is configured to receive the responses and to automatically generate an entry decision regarding entry to the facility by the potential entrant based on the responses. The electronic medical evaluation system is configured to automatically send an indication of the entry decision to the interface, and potentially the same or similar indications to one or more additional interfaces, e.g., an additional display at the same facility, to enable entry to the facility to be controlled.

According to one aspect, the electronic medical evaluation system and the potential entrant interface may be embedded within or hosted by a mobile electronic device that is usable by a potential entrant to display a questionnaire and to provide responses to the questionnaire. According to another aspect, the electronic medical evaluation system may be embedded or hosted remotely in or by another electronic device separate from a mobile electronic device hosting the potential entrant interface, such as a remote computer, server, or other electronic device. According to yet another aspect, either or both of the electronic medical evaluation system and the potential entrant interface may be embedded or hosted in or by an electronic device at or near the facility to which entry is requested. According to yet another aspect, one or more questionnaires may be hosted separately from the electronic medical evaluation system and/or the potential entrant interface in a mobile or other electronic device which may include a remote computer, server, or other electronic device which may be located at, near, or remote from the facility to which entry is requested.

According to one aspect, the electronic medical evaluation system comprises a plurality of questionnaires. The electronic medical evaluation system is configured to select the questionnaire to send to the potential entrant interface for display from the plurality of questionnaires based on various factors. The factors may include, for example, a type or identity of the potential entrant, a type or identity of the facility to which entry is requested, the purposes for which entry to the facility is requested, and/or a type or identity of a job of the potential entrant.

According to another aspect, a code may be posted at or near the facility to which entry is requested. The code may encode one or more of the factors for selecting the questionnaire. The interface may comprise a portable device and may be configured to read the code. The electronic medical evaluation system may be configured to select the questionnaire to send to the potential entrant interface from the plurality of questionnaires based on the factors encoded by the code.

According to another aspect, the electronic medical evaluation system comprises a storage adapted to store a plurality of records corresponding to a plurality of potential entrants. Each record may comprise one or more of a potential entrant identifier, the decision regarding entry to the facility, a facility identifier, a date of the decision, a time of the decision, a questionnaire identifier, and an indication of subsequent status of the potential entrant.

According to another aspect, the indication of the entry decision comprises or corresponds to an entry decision presentation by the potential entrant interface. The entry decision presentation may comprise a visually perceptible element such as a color or other visually perceptible element that may be displayed, an auditory element, a tactile element, or a combination thereof. The indication of the entry decision may comprise a plurality of different indications to enable manual control of entry to the facility. One indication may correspond to an entry decision clearing the potential entrant for entry. A second indication may correspond to an entry decision not clearing the potential entrant for entry. A third indication may correspond to a decision to delay the entry decision. According to another aspect, the indication of the entry decision may comprise a machine readable code indicative of the entry decision to enable automated control of entry to the facility. According to another aspect, the electronic medical evaluation system is configured to generate a request for medical evaluation for the potential entrant depending on the entry decision.

The example medical screening system may be used according to an example method to medically screen a potential entrant to a facility before the potential entrant enters the facility. According to the example method, the electronic medical evaluation system receives a request from the potential entrant interface for a questionnaire to be sent. The electronic medical evaluation system sends the questionnaire to the interface. The questionnaire is displayed on the interface. The potential entrant provides responses to the questions of the questionnaire. The responses are received by the electronic medical evaluation system and the electronic medical evaluation system automatically generates an entry decision regarding entry to the facility by the potential entrant based on the responses. The electronic medical evaluation system automatically sends an indication of the entry decision to the interface, and the potential entrant interface provides a corresponding entry decision presentation comprising one or more visual, auditory, or tactile perceptible elements to enable entry to the facility by the potential entrant to be controlled. Depending on the entry decision, a request for medical evaluation of the potential entrant may be generated.

There has thus been outlined, rather broadly, some of the embodiments of the medical screening system and method in order that the detailed description thereof may be better understood, and in order that the present contribution to the art may be better appreciated. There are additional embodiments of the medical screening system and method that will be described hereinafter and that will form the subject matter of the claims appended hereto. In this respect, before explaining at least one embodiment of the medical screening system and method in detail, it is to be understood that the medical screening system and method is not limited in its application to the details of construction or to the arrangements of the components set forth in the following description or illustrated in the drawings. The medical screening system and method is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

Example embodiments will become more fully understood from the detailed description given herein below and the accompanying drawings, wherein like elements are represented by like reference characters, which are given by way of illustration only and thus are not limitative of the example embodiments herein.

FIG. 1 is a block diagram of a medical screening system and method in accordance with an example embodiment.

FIG. 2 is an example data entry device displaying a registration/log-in page of a potential entrant interface of a medical screening system and method in accordance with an example embodiment.

FIG. 3 is an example data entry device displaying an example pre-entry questionnaire of a potential entrant interface of a medical screening system and method in accordance with an example embodiment.

FIG. 4 is an example data entry device displaying another example pre-entry questionnaire of a potential entrant interface of a medical screening system and method in accordance with an example embodiment.

FIG. 5 is an example data entry device displaying another example pre-entry questionnaire of a potential entrant interface of a medical screening system and method in accordance with an example embodiment.

FIG. 6 is an example data entry device displaying another example pre-entry questionnaire of a potential entrant interface of a medical screening system and method in accordance with an example embodiment.

FIG. 7 is an example data entry device displaying an example pre-entry questionnaire summary page of a potential entrant interface of a medical screening system and method in accordance with an example embodiment.

FIG. 8 is an example data entry device displaying an example pre-entry questionnaire disclaimer page of a potential entrant interface of a medical screening system and method in accordance with an example embodiment.

FIG. 9 is an example data entry device displaying an example entry decision presentation page of a potential entrant interface of a medical screening system and method comprising a visual, audio, or tactile element in accordance with an example embodiment.

FIG. 10 is an example data entry device displaying another example entry decision presentation page of a potential entrant interface of a medical screening system and method comprising a visual, audio, or tactile element in accordance with an example embodiment.

FIG. 11 is a display of an example entrant status and records screen of an electronic medical evaluation system of a medical screening system and method in accordance with an example embodiment.

FIG. 12A is a display of another example entrant status and records screen of an electronic medical evaluation system of a medical screening system and method in accordance with an example embodiment.

FIG. 12B is a display of another example entrant status and records screen of an electronic medical evaluation system of a medical screening system and method in accordance with an example embodiment.

FIG. 13 is a flowchart illustrating an example method of operation of a medical screening system and method in accordance with an example embodiment.

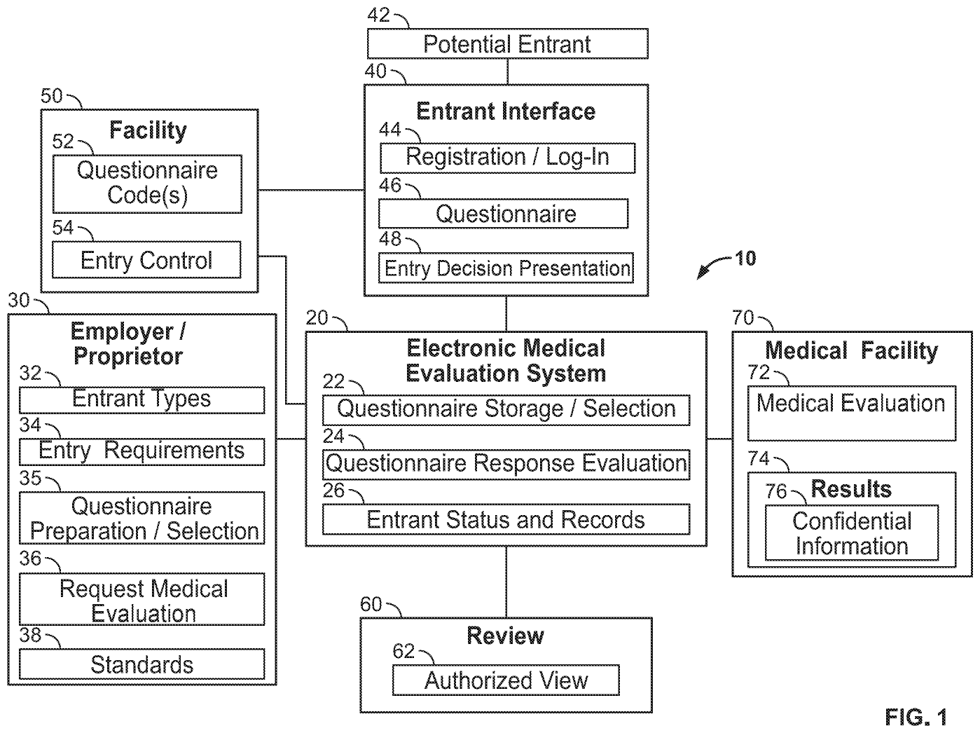

### DETAILED DESCRIPTION

### A. Overview

An example medical screening system and method 10 screens potential entrants 42 requesting entry to facilities 50 for medical conditions prior to entry. The example medical screening system and method 10 generally includes an electronic medical evaluation system 20 and a potential entrant interface 40.

The electronic medical evaluation system 20 preferably comprises a centralized computing system for receiving, sending, processing, and sharing information and data with the potential entrant interface 40, employers and proprietors 30, facilities 50, a review unit 60, and medical facilities 70. The electronic medical evaluation system 20 preferably comprises questionnaire storage and selection 22, questionnaire response evaluation 24, and entrant status and records 26 components.

The questionnaire storage and selection component 22 is adapted and configured to store a plurality of questionnaires 46 and to automatically select an appropriate questionnaire 46 in response to a request from the potential entrant interface 40 based on a plurality of factors related to a potential entrant 40, a facility 50 to which entry is requested, and others. The plurality of questionnaires 46 may include questionnaires prepared by employers 30 of potential entrants 42, proprietors 30 of the facilities 50, and/or an entity controlling the operation of the electronic medical evaluation system 20.

The potential entrant interface 40 is preferably implemented as a downloadable application and is embedded within or hosted and presented by a portable electronic device 80. Alternatively, the potential entrant interface 40 may be embedded in or hosted by an electronic device at or near a facility 50 to which entry is requested or another electronic device. The potential entrant interface 40 is adapted and configured to receive a selected questionnaire 46 from the electronic medical evaluation system 20 and to display the questionnaire 46. The displayed questionnaire 46 elicits from a potential entrant 42 responses, information, and data concerning the potential entrant's identity and medical condition, including for example exposure to an infectious disease, symptoms, etc.

Some or all of the components of the electronic medical evaluation system 20 may be embedded within or hosted by the same mobile or other electronic device as the potential entrant interface 40, or in another separate electronic device such as a separate centralized computer or server. Also similarly, the questionnaires 46 may be embedded in or hosted by the same electronic device as the other components of the electronic medical evaluation system 20 and/or the potential entrant interface 40, or may be embedded in or hosted by a separate electronic device.

The responses, information, and data provided by the potential entrant 42 in response to the selected questionnaire 46 are received from the potential entrant interface 40 by the electronic medical evaluation system 20. The questionnaire response evaluation component 24 automatically and algorithmically processes and evaluates the responses, information, and data and automatically generates an entry decision therefrom. The responses, information, and data, the entry decision, and the medical and entry status of the potential entrant 42 are stored in the entrant status and records 26 component.

The electronic medical evaluation system 20 sends an indication of the entry decision to the potential entrant interface 40. In response, the potential entrant interface 40 provides an entry decision presentation 48 comprising one or more visual, auditory, or tactile elements and corresponding to and reflecting the entry decision. The visual, auditory, or tactile elements may comprise, for example, a green box indicating a cleared for entry decision, and a red box indicating a not cleared for entry decision. Entry control 54 at the facility 50, for example security, an entrance attendant, or another entry controller, controls entry by the potential entrant 42 to the facility 50 based on the entry decision presentation 48.

The electronic medical evaluation system 20 provides responses, information, and data provided by potential entrants 42 in response to questionnaires 46 to the review unit 60 for review. The review unit 60 reviews the same and excludes confidential information subject to privacy laws or other exclusion criteria, or includes such confidential information in authorized views 62 that are accessible only by authorized persons. Employers and proprietors 30 may be provided real-time access to the records of potential entrants 42 stored in the entrant status and records 26 component except for the confidential or excluded information.

In the case of responses and information resulting in a not cleared for entry decision, the review unit 60 may also recommend or propose medical evaluation of the affected potential entrant 42. The electronic medical evaluation system 20 may provide a notice to the associated employer or proprietor 30, receive a request for medical evaluation from the employer or proprietor 30, and provide the request to a medical facility 70.

The medical facility 70 performs the medical evaluation of the potential entrant 42, and reports the results to the electronic medical evaluation system 20. The review unit 60 may review the results and redact confidential information or include it in authorized views 62. The electronic medical evaluation system 20 updates and maintains the records for the potential entrants 42 in the entrant status and records 26 component.

The example medical screening system and method 10 is capable of screening a plurality of potential entrants 42 associated with a plurality of employers and proprietors 30 for entry to a plurality of facilities 50 for medical conditions and for providing a plurality of entry decisions for the potential entrants 42 and facilities 50. The example medical screening system and method 10 is also capable of storing, controlling access to, updating, and maintaining the records for the plurality of potential entrants 42 including their entry and medical status.

### B. Electronic Medical Evaluation System

The electronic medical evaluation system 20 of the medical screening system and method 10 preferably comprises a centralized system for sending, receiving, processing, storing, communicating, and sharing medical and other data and information regarding potential entrants 42 to facilities 50. The electronic medical evaluation system 20 may be comprised of the components and functionality of the electronics medical evaluation system shown and described in U.S. Patent Application Pub. No. US 2016/01804843 A1 and U.S. Patent Application Pub. No. US 2016/0180292 A1, both assigned to the assignee of the present application, and both incorporated herein by reference in their entirety as if set forth herein. The electronic medical evaluation system 20 also comprises the components and functionality described herein.

The electronic medical evaluation system 20 is coupled to and adapted to communicate data and information with one or more employers/proprietors 30, one or more potential entrant interfaces 40, one or more facilities 50, a review unit 60, and one or more medical facilities 70. More particularly, the electronic medical evaluation system 20 is adapted and configured to receive, send, process, and store data and information to automatically medically screen a potential entrant 42 prior to entry to a facility 50, generate an entry decision whether the potential entrant 42 is cleared or not cleared to enter the facility 50, and communicate an indication of that decision to enable entry to the facility 50 to be controlled. The electronic medical evaluation system 20 is also adapted and configured to generate and communicate a notice regarding needed, proposed, or recommended evaluation of a potential entrant 42, and to receive and communicate a request for medical evaluation of a potential entrant 42 depending on the entry decision.

The electronic medical evaluation system 20 described herein may be embedded in or hosted by an electronic device such as a computer, server, or similar electronic device. In some embodiments, one or more components of the electronic medical evaluation system 20 may be embedded or hosted in the same electronic device in which the potential entrant interface 40 is embedded or hosted. That electronic device may comprise the mobile electronic device 80 described herein. In other embodiments, one or more components of the electronic medical evaluation system 20 may be embedded or hosted in one or more electronic devices that are separate and/or remote from the electronic device in which the potential entrant interface 40 is embedded or hosted, such as a remote computer or server.

The electronic medical evaluation system 20 preferably comprises a questionnaire storage and selection 22 component. The questionnaire storage and selection 22 component preferably comprises a storage for storing a plurality of different questionnaires 46 and logic and/or programming adapted and configured for selecting an appropriate questionnaire 46 from the plurality of stored questionnaires 46. In some embodiments, the questionnaire storage and selection component 22 and questionnaires 46 may be embedded in or hosted by the same electronic device as one or more of the other components of the electronic medical evaluation system 20 and/or the potential entrant interface 40. In one embodiment, that electronic device may comprise the mobile electronic device 80 described herein. In other embodiments, the questionnaire storage and selection component 22 and questionnaires 46 may be embedded or hosted in a separate and/or remote electronic device from the electronic devices in which the potential entrant interface 40 and/or other components of the electronic medical evaluation system 20 are embedded or hosted.

One or more of the plurality of stored questionnaires 46 may be received by the electronic medical evaluation system 20 from one or more employers 30 of potential entrants 42, or from one or more proprietors 30 of facilities 50, or both. One or more of the plurality of stored questionnaires 46 also may comprise standard questionnaires prepared by the entity that controls the operation of the electronic medical evaluation system 20. Further, one or more of the plurality of stored questionnaires 46 may comprise such standard questionnaires 46 modified by an employer or proprietor 30. For example, an employer 30 may modify a standard questionnaire 46 provided by the operator of the electronic medical evaluation system 20 to include questions related to unique or specific requirements of the employer 30 based on the employer's business, regulations relating to the employer's business, entrant type, employee or employee type, job or job type, etc. Similarly, a proprietor 30 of a facility 50 may modify a standard questionnaire 46 provided by the operator of the electronic medical evaluation system 20 to include questions related to unique or specific requirements of the proprietor 30 or facility 50 similar to those identified above for an employer 30.

The plurality of stored questionnaires 46 may comprise any combination of questionnaires 46 prepared and provided by a plurality of employers/proprietors 30, standard questionnaires prepared by the entity controlling operation of the electronic medical evaluation system 20, and such standard questionnaires modified by a plurality of employers/proprietors 30. The plurality of stored questionnaires 46 may also comprise questionnaires for a plurality of different employers/proprietors 30 and for a plurality of different facilities 50.

The questionnaire storage/selection component 22 is preferably adapted and configured to select an appropriate questionnaire 46 from the plurality of stored questionnaires 46 for the electronic medical evaluation system 20 to communicate or send to a potential entrant interface 40 to determine whether a potential entrant 42 to a facility 50 should be cleared and allowed entry or not cleared and denied entry. The questionnaire storage/selection component 22 is preferably adapted and configured to select a questionnaire 46 that is appropriate for a potential entrant 42, and that meets the requirements of an employer/proprietor 30 and of the facility 50 to which entry is requested. The selection may be based on one or a combination of different factors.

The factors may include, for example, a type or identity of the potential entrant 42, a type or identity of the facility 50 to which entry is requested, a type or identity of a job of the potential entrant 42, or a combination thereof, among others. Thus, for example, the questionnaire storage/selection component 22 may be adapted and configured to select one questionnaire 46 for a potential entrant 42 that is an employee, a different questionnaire 46 for an independent contractor, another different questionnaire 46 for a vendor or supplier, and yet another different questionnaire 46 for a customer. Yet other different questionnaires 46 may be selected for a potential entrant 42 that is requesting entry for the first time, a returning potential entrant 42, a potential entrant 42 that has previously been cleared and allowed entry, and a potential entrant 42 that has previously been not cleared and denied entry.

Similarly, for example, the questionnaire storage/selection component 22 may be adapted and configured to select one questionnaire 46 where the facility 50 to which entry is requested is a restaurant, or a common carrier, another questionnaire 46 where the facility 50 is a manufacturing plant or warehouse, and yet another questionnaire 46 where the facility 50 is a stadium or arena. Also for example, questionnaire storage/selection component 22 may be adapted and configured to select one questionnaire 46 when the potential entrant's job may not involve close contact with others, for example a warehouse position, another when the potential entrant's job may involve a relatively greater amount of contact with others, such as an office position, and yet another when the potential entrant's job may involve close contact with numerous other persons, such as a retail associate, usher, ticket taker at a stadium, or chef or waiter in a restaurant.

The factors to be used to select a questionnaire 46 may be specified by an employer 30, the proprietor 30 of a facility 50 to which entry is requested, the entity in control of the operation of the electronic medical evaluation system 20, or a combination thereof. Further, and as described further below, one or more of the factors may be encoded in and indicated by a code 52, for example a QR or bar code, which may be provided at or by a facility 50 to which entry is requested. Alternatively, the code 52 could encode a selection of a questionnaire 46 or a questionnaire itself rather than factors for selecting a questionnaire 46. The electronic medical evaluation system 20 may receive the code 52 via the potential entrant interface 40, interpret or decode it, and the questionnaire storage/selection component 22 may be adapted and configured to use the decoded factors or the decoded questionnaire selection to select the appropriate questionnaire for the facility 50. Alternatively, the code 52 may be decoded by the potential entrant interface 40 described further below, before being sent to the electronic medical evaluation system 20.

The electronic medical evaluation system 20 is adapted and configured to send the selected questionnaire 46 to the potential entrant interface 40 for display. The electronic medical evaluation system 20 is also adapted and configured to receive from the potential entrant interface 40 the responses, information, and data provided by a potential entrant 42 in response to the questions included in the displayed questionnaire 46.

The electronic medical evaluation system 20 preferably includes a questionnaire response evaluation 24 component. The questionnaire response evaluation component 24 preferably comprises logic and/or programming that is adapted and configured to automatically evaluate the responses, information, and data provided by the potential entrant 42 to the questions included in a questionnaire 46 and to automatically generate an entry decision based thereon whether the potential entrant 42 is cleared and allowed entry to the facility 50, or is not cleared and is denied entry to the facility 50. The questionnaire response evaluation 24 may comprise one or more suitable artificial intelligence (AI) and/or other algorithms that are adapted and configured to process and evaluate the responses, information, and data provided by a potential entrant 42 and to generate the entry decision based thereon. In an embodiment, a human reviewer may also review the responses, information, and data, as well as the algorithms and entry decisions and may make necessary changes to the algorithms and/or to entry decisions as necessary for accuracy. Even if a human reviewer is employed in some embodiments, however, the evaluation and entry decision generation process is automatic insofar as the employers and proprietors 30 are concerned.

The questionnaire response evaluation 24 component is preferably adapted and configured to send an indication of the entry decision to the potential entrant interface 40 from which the questionnaire responses were received to enable entry to the facility 50 by the potential entrant 42 to be controlled. The questionnaire response evaluation 24 component may also be adapted and configured to send an indication of the entry decision to other interfaces and/or other electronic devices, for example other interfaces presented by screens of other electronic devices located in or around the facility 50. The indication of the entry decision is preferably adapted and configured to be used by the potential entrant interface 40 to provide an entry decision presentation 48 corresponding to and reflecting the entry decision. The entry decision presentation 48 preferably comprises a visually perceptible element that is displayable by the potential entrant interface 40. For example, the visually perceptible element may comprise a first color corresponding to and reflecting an entry decision clearing the potential entrant 42 for entry to the facility 50 and a second color corresponding to and reflecting an entry decision not clearing the potential entrant 42 for entry to the facility 50. In one embodiment, the visually perceptible element may comprise a third color corresponding to and reflecting a decision to delay making an entry decision.

The entry decision presentation 48 may also comprise other visually perceptible elements, including for example, other colors, blinking elements, and scrolling elements. The entry decision presentation 48 may also comprise one or more non-visually perceptible elements, such as auditory and tactile elements like sounds, vibration, etc. The entry decision presentation 48 may comprise any combination of visually perceptible and non-visually perceptible elements desired to indicate the nature of the entry decision and to differentiate between different entry decisions.

It will be appreciated that the entry decision presentation 48 provided by the potential entrant interface 40 and corresponding to and reflecting the entry decision generated by the electronic medical evaluation system 20 enables an entry control 54 at the facility 50 to easily and quickly manage and control entry to the facility 50 manually, for example by a security guard or other attendant stationed at or near an entrance. Alternately or in addition, the entry decision presentation 48 may comprise a form that enables the entry control 54 to automatically manage and control entry. For example, the entry decision presentation 48 may comprise a machine readable code that corresponds to the entry decision, and the entry control 54 may comprise a reader located at or near an entrance that reads the machine readable code and automatically unlocks and/or opens the entrance when the code corresponds to a cleared entry decision.

The electronic medical evaluation system 20 also preferably comprises an entrant status and records 26 component. The entrant status and records 24 preferably comprises a storage for a plurality of records corresponding to a plurality of potential entrants 42 including their entry status, i.e., cleared or not cleared for entry to one or more facilities 50. The storage may be the same storage used by the questionnaire storage and selection component 22 for the storage of questionnaires 46 or a separate storage. The entrant status and records 24 also preferably comprises logic and/or programming that is adapted and configured to enable records to be searched, selected, reviewed, and edited for particular potential entrants 42.

Preferably the entrant status and records 24 component is adapted and configured to be accessible by employers and proprietors 30 and to permit and enable them to search, locate, identify, and review the records corresponding to their associated potential entrants 42, e.g., their employees, contractors, or other types of potential entrants 42 as described herein. For example, the entrant status and records 24 component may comprise a secure remote login for registered employers and proprietors 30. However, some of the data or information in the records of potential entrants 42 may be legally protected against disclosure to the employers and proprietors 30 by privacy laws such as the Health Insurance Portability and Accountability Act (HIPAA) or for other reasons. Accordingly, the electronic medical evaluation system 20 preferably comprises suitable filters, firewalls, or other means adapted and configured to prevent protected data and information of potential entrants 42 from being sent to, viewed by, and/or accessed by employers and proprietors 30, and by any other persons not authorized to access and view the protected data and information.

The electronic medical evaluation system 20 is also preferably adapted and configured to be accessible by a review unit 60 and/or to communicate, send, and receive data and information to and from the review unit 60. Similar to access by employers and proprietors 30, access may be provided to the review unit 60 by a secure login. The data and information may include responses to questionnaires 46 by potential entrants 42, including potential entrant 42 identifying information, job information, and medical information. The data and information may also include medical evaluations, treatments and/or tests performed or to be performed for a potential entrant 42 by a medical facility 70 and/or the results thereof.

The electronic medical evaluation system 20 is adapted and configured to protect the privacy rights of potential entrants 42 in connection with the review unit 60. For example, the review unit 60 may identify a portion of the data and information received from the electronic medical evaluation system 20, including for example certain responses to questionnaires 46 and certain test results 112, as confidential information that is not to be accessible by or shared with an employer or proprietor 30. The review unit 60 may redact that information or selectively include it in an authorized view 62 that is only accessible to authorized persons and that is not generally accessible to employers or proprietors 30 of the potential entrants 42. The electronic medical evaluation system 20 may be adapted and configured to receive information and data from the review unit 60 and to implement the appropriate redactions and/or authorized views 62 in the potential entrant status and records 26 component. This preserves the employer or proprietor's 30 real-time access to the information and data to which it is legally allowed access while protecting the privacy rights of the potential entrants 42.

The electronic medical evaluation system 20 and/or the review unit 60 may be adapted and configured to generate a notice or recommendation regarding medical evaluation of a potential entrant 42 based on the potential entrant's 42 responses to the questionnaire 46, the entry decision by the questionnaire response evaluation component 24, and other information and data regarding the potential applicant 42, as well as entry requirements 34 of an employer or proprietor 30. If the notice or recommendation originates from the review unit 60, it is preferably communicated or sent to the electronic medical evaluation system 20 for communication to the employer or proprietor 30 associated with the potential entrant 42. The electronic medical evaluation system 20 may be adapted and configured to provide the notice or recommendation to an employer or proprietor 30 as a message, for example by email, text or otherwise, or by including it in the potential entrant's 42 record in the entrant status and records 26, or otherwise.

The electronic medical evaluation system 20 is preferably adapted and configured to receive from employers and proprietors 30 a request for medical evaluation 36 of a potential entrant 42. For example, the request 36 may result from a decision by the electronic medical evaluation system 20 that a potential entrant 42 is not cleared and not allowed to enter a facility 50. It may also result from a notice or recommendation by the electronic medical evaluation system 20 regarding proposed medical evaluation for a potential entrant 42.

The electronic medical evaluation system 20 is preferably adapted and configured to respond to a request 36 for medical evaluation received from an employer or proprietor 30 by communicating or sending a request for medical evaluation 72 to a medical facility 70. The electronic medical evaluation system 20 is also preferably adapted and configured to update the record of the potential entrant 42 that is to receive the medical evaluation 72 in the entrant status and records 26 appropriately, including updating the status of the potential entrant 42 and the status date.

The electronic medical evaluation system 20 is preferably adapted and configured to receive from the medical facility 70 that performs the medical evaluation 72 various data and information concerning the potential entrant 42. The medical facility 70 may be provided a secure login to the electronic medical evaluation system 20 for that purpose. The data and information may reflect or be related to, for example, the medical evaluation 72 performed, the results 74, and/or other information concerning diagnoses, prognoses, medical recommendations, and other medical data and information. Some of the information and data may comprise confidential information and data 76 that is legally protected by privacy laws against disclosure as described herein. However, confidential information and data 76 may or may not be identified as such by the medical facility 70 that performed the medical evaluation 72.

The electronic medical evaluation system 20 is preferably adapted and configured to respond to the receipt of the information and data from a medical facility 70 by making the information and data accessible to the review unit 60 in the same manner as described above for appropriate redaction or exclusion from an authorized view 62. Also as described above, the electronic medical evaluation system 20 is preferably adapted and configured to receive information and data from the review unit 60, implement the appropriate redactions and/or authorized views 62, and update the potential entrant's 42 record in the potential entrant status and records 26 with the information and data received from the medical facility 70, including updating the potential entrant's 42 status and status date. The electronic medical evaluation system 20 may also be adapted and configured to generate and send a notice to the employer or proprietor 30 that requested 36 the medical evaluation 72.

The electronic medical evaluation system 20 may also be adapted and configured to ensure that a medical facility 70 performing medical evaluation 72 of a potential entrant 42 adheres to standards 38 of the employer or proprietor 30 that requested 36 the medical evaluation. The review unit 60 may review the medical evaluation, treatment and/or tests 72 performed and the results 74 obtained to ensure that the standards 38 have been complied with. Based on the review, the review unit 60 may accept, decline, or request additional information from the medical facility 76 via the electronic medical evaluation system 20. The electronic medical evaluation system 20 is thus adapted and configured to ensure that consistent medical care is provided in accordance with the standards 38 for all potential entrants 42 associated with an employer or proprietor 30 regardless of the medical facility 70 providing the evaluation.

The electronic medical evaluation system 20 thus may provide a centralized or distributed system by which a plurality of potential entrants 42 associated with a plurality of different employers and proprietors 30 and requesting entry to a plurality of different facilities 50 can be medically screened and entry decisions automatically made and communicated substantially in real-time and without burdening the employers and proprietors 30 associated with the potential entrants 42. The electronic medical evaluation system 20 also may provide a centralized or distributed repository of records including medical and entry status for a plurality of potential entrants 42 associated with a plurality of employers and proprietors 30 and a plurality of facilities 50. The electronic medical evaluation system 20 also may provide a centralized or distributed system by which a plurality of employers and proprietors 30 are able to track the medical screening and facility entry status of a plurality of employees and other potential entrants 42 to a plurality of facilities 50. The electronic medical evaluation system 20 also may provide controlled access to such records based on the associations between potential entrants 42, and employers and proprietors 30, and in accordance with applicable privacy laws and requirements. The electronic medical evaluation system 20 also may provide a centralized or distributed system by which medical evaluation can be requested and performed by a plurality of medical facilities 70 in a plurality of locations for a plurality of potential entrants 42 based on medical screening and entry decisions made for such potential entrants 42 with respect to a plurality of facilities 50 in different locations, and by which the records of such potential entrants 42 can be updated and maintained accordingly.

The electronic medical evaluation system 20 may also allow for audits and tracking of a plurality of potential entrants 42 associated with a plurality of different employers and proprietors 30 who sought entry to a plurality of facilities 50 in a plurality of different locations. For example, the electronic medical evaluation system 20 provides the ability for the records of all potential entrants 42 that were denied entry to a facility 50 over a period of time due to exposure to a particular infectious disease to be searched, correlated, and the potential entrants 42 identified. These records can be correlated with the records of other potential entrants 42 that were cleared and allowed entry to the facility 50 during the same time period in order to identify and track the potential exposure of others to the infectious disease, i.e., provide contact tracing. This enables employers and proprietors 30 to take appropriate actions, for example notifying persons who were potentially exposed, performing appropriate sanitation actions to affected facilities 50, and/or taking steps to quarantine the affected persons and/or facilities 50.

### C. Questionnaires

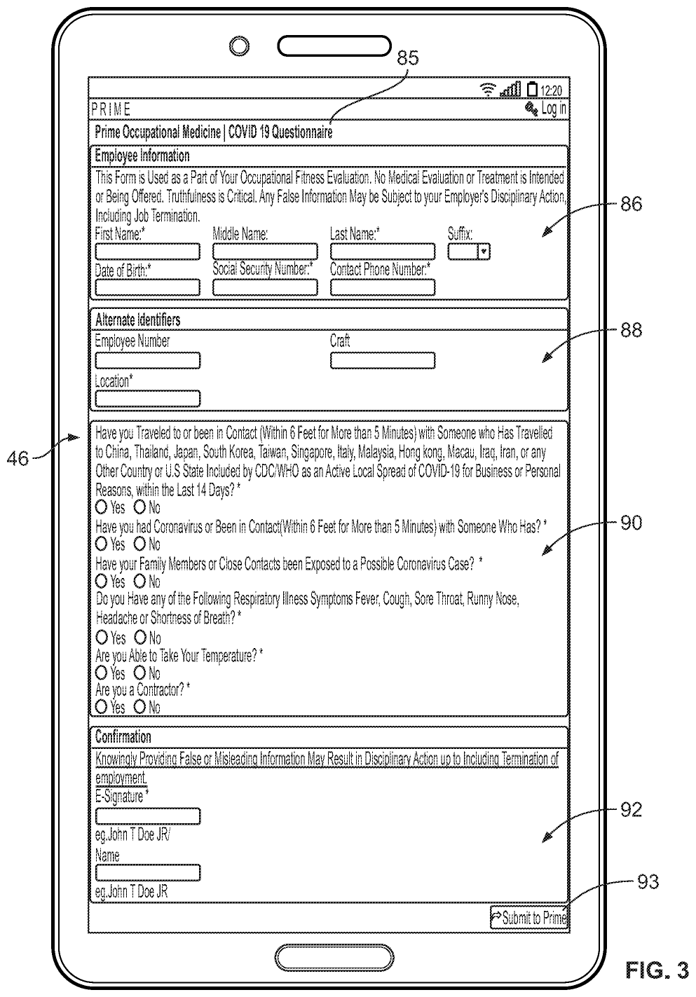

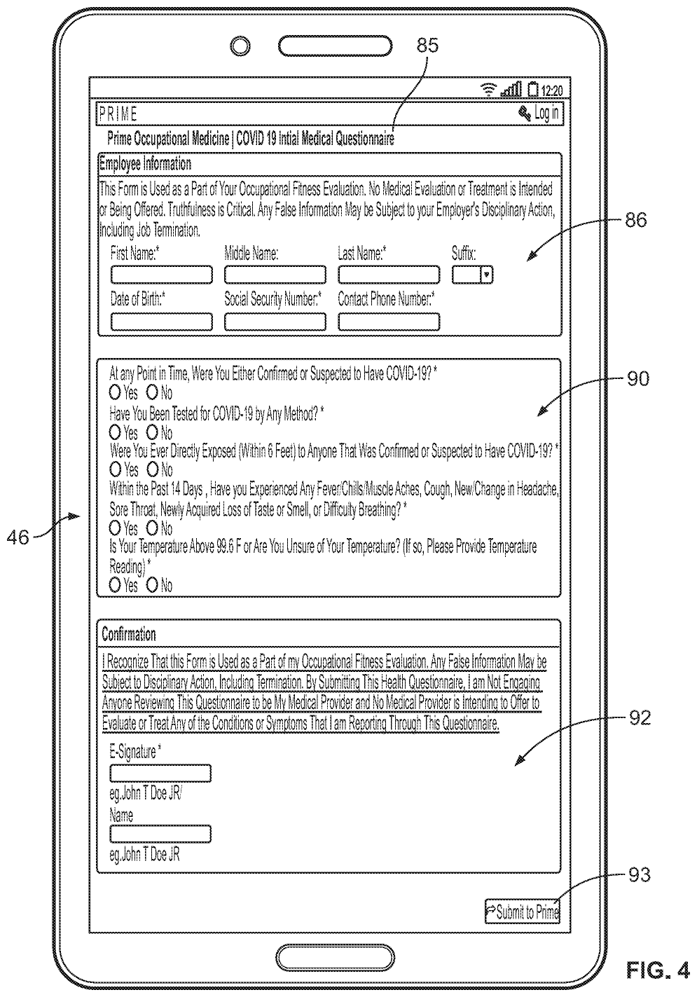

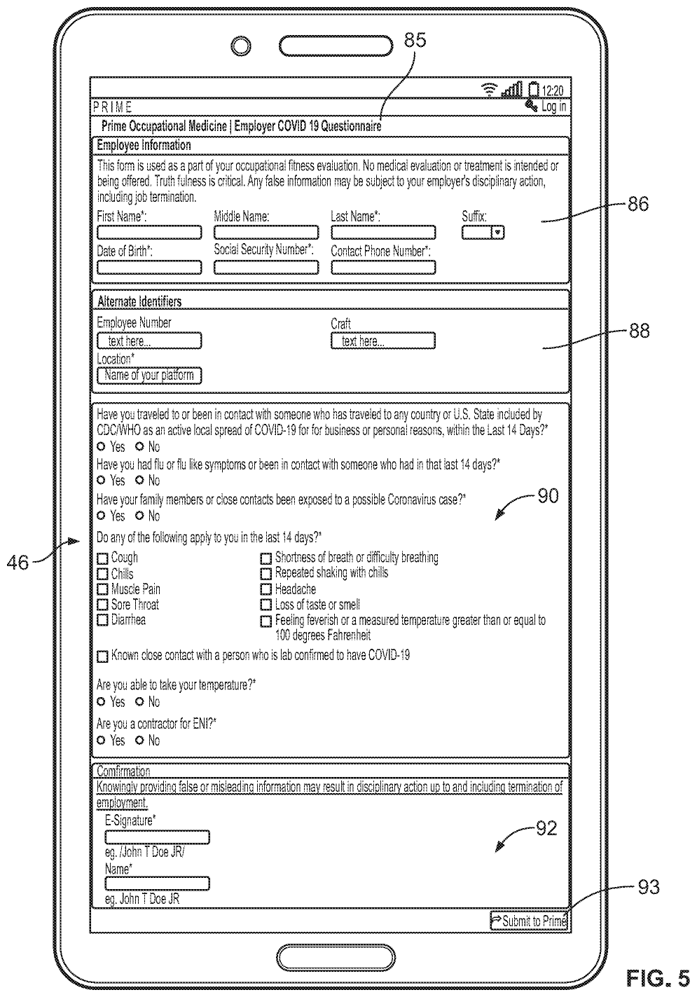

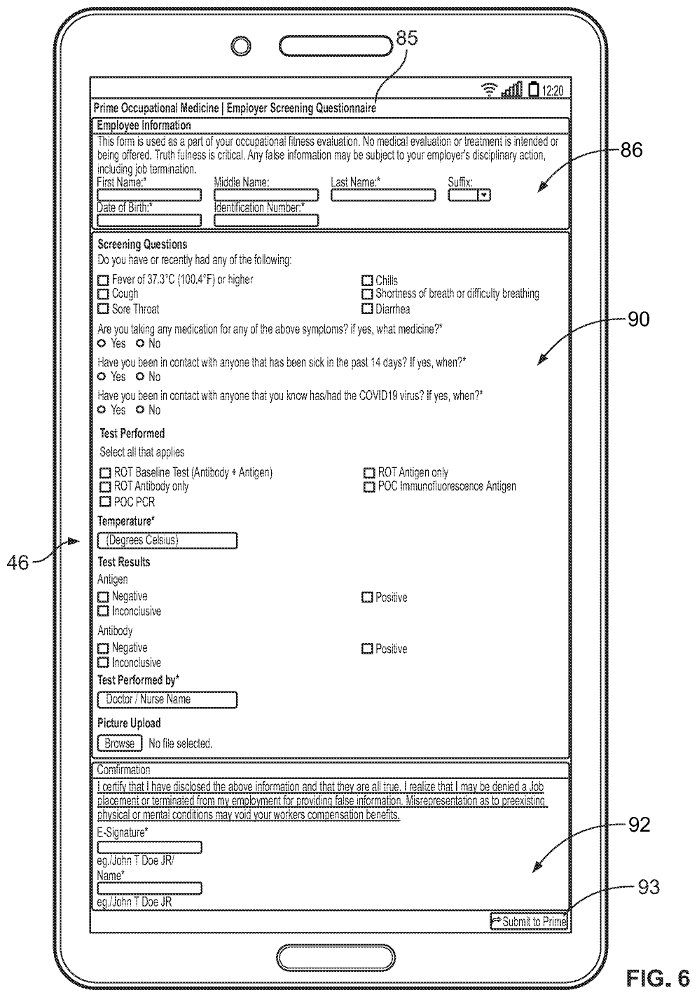

Several example questionnaires 46 are illustrated in FIGS. 3-6. A questionnaire 46 may comprise any form or template comprising questions, information or data entry fields, check-boxes, and/or other devices or tools that are adapted, arranged, and configured to elicit responses, information, and data for identifying and medically screening a potential entrant 42 to a facility 50 in order to make an entry decision whether the potential entrant 42 is cleared or not cleared for entry to the facility 50. The questionnaire 46 is preferably in a form that is displayable in, on, or as part of the potential entrant interface 40. The questionnaire 46 is preferably in a form that is interactive so that a potential entrant 42 may provide responses to questions, make selections, and enter requested information and data using the potential entrant interface 40.

The questionnaires 46 may be embedded in or hosted by the same electronic device as other components of the electronic medical evaluation system 20 and/or the potential entrant interface 40. That electronic device may comprise the mobile electronic device 80 or another mobile or non-mobile electronic device. The questionnaires 46 also may be embedded in or hosted by a separate and perhaps remote electronic device from the electronic device on which other components of the electronic evaluation system 20 and/or the potential entrant interface 40 are embedded or hosted.

Each questionnaire 46 preferably includes a questionnaire identifier 85. The questionnaire identifier 85 is preferably displayable with the questionnaire 46 with which it is associated in, on, or as part of the potential entrant interface 40. The questionnaire identifier 85 may comprise a title or other description of the questionnaire 46 with which it is associated, for example “COVID 19 Questionnaire,” “COVID-19 Initial Medical Questionnaire,” or “[Employer Name] COVID19 Questionnaire.”

A questionnaire 46 may comprise multiple sections and each section may comprise multiple questions, selection boxes, data entry fields, etc. A questionnaire 46 may comprise, for example, a section for eliciting information identifying a potential entrant 42. In that section, entrant identification fields 86 may elicit answers and information including, for example, the name, date of birth, social security number, telephone number, and other information for identifying the potential entrant 42.

Another section may elicit identifying information that is specific to an employee and the employee's job. For example, employee identification fields 88 may elicit answers and information including, for example, an employee number, employee craft or job title or number, location, and other information for identifying the employee and the employee's job functions and locations. It will be appreciated that questionnaires 46 for screening potential entrants 42 that are not employees, e.g., customers, vendors, fans, guests, visitors and the like, need not include such fields.

Another section may elicit information concerning the medical condition of the potential entrant 42. For example, a plurality of medical questions 90 may be presented in the form of a plurality of data entry fields, check boxes, selections, and the like. The medical questions 90 may elicit answers, information, and data concerning the potential entrant's current medical condition, symptoms, exposure history, etc. It will be appreciated that a wide variety of different answers, information, and data may be elicited depending on the potential entrant 42, facility 50 to which entry is requested, and the particular issues of concern to employers and proprietors 30. It will also be appreciated that different questionnaires 46 may comprise different sets and/or types of questions which may overlap to some degree.

By way of more specific examples, the medical questions 90 may elicit information such as whether the potential entrant 42: has been confirmed or suspected to have a condition or infectious disease, e.g., Covid-19, Ebola, has been tested for a particular infectious disease or type of infectious disease, has been exposed to a particular infectious disease or type of disease or has engaged in conduct that may result in exposure, is experiencing or has experienced particular identified symptoms within a certain period of time, and the nature and values associated with such symptoms, e.g., temperature. The medical questions 90 may also elicit information such as whether potential entrant 42: has recently traveled to a specified region or area within a specified period of time, has family or contacts who have been exposed to a particular infectious disease. Still further, the medical questions 90 may elicit information such as whether the potential entrant 42: is taking any medication for reported symptoms, has had particular identified tests performed, the results of the tests, and when and by whom the tests were performed. The medical questions 90 section of the questionnaire 46 may also include one or more buttons or other selectors to enable the potential entrant 42 to select and upload test results.

Another section may elicit a confirmation or verification 92 that the answers and information provided by the potential entrant 42 are true and accurate. The confirmation or verification 92 may comprise, for example, a signature, such as an e-signature, of the potential entrant 42. This section may also include a submit selector 93, which may comprise a button, icon or the like that is selectable by the potential entrant 42. Selection of the submit selector 93 preferably indicates to the potential entrant interface 40 that the potential entrant 42 has completed responding to the questionnaire 46 and is ready to continue to the next step of the process for submitting the responses, information, and data to the electronic medical evaluation system 20 for processing.

It will be appreciated that various questionnaires 46 may comprise more, fewer and/or different sections and questions, and will elicit more, less, or different answer and information than the questionnaires 46 described above, which are given by way of example only. The sections, questions, and answers and information elicited in any particular questionnaire will depend on a variety of factors, which may include the type and/or identity of the potential entrant 42, the entry requirements of an employer/proprietor 30 and/or of the facility 50 to which entry is requested, the type or identity of the facility 50 to which entry is requested, the employee status and the type and/or identity of the job of the potential entrant 42, or a combination thereof, among others. Thus, for example, questionnaires 46 may have different sections and questions depending on whether they are intended to screen employees, independent contractors, vendors or suppliers, customers, guests or visitors. They may have different sections and questions depending on whether the potential entrant 42 to be screened is an office worker, warehouse worker, retail associate, ticket taker, etc. They may also have different sections and questions depending on whether the facility 50 to which entry is requested is a warehouse, office, restaurant, stadium or arena, etc. Still further, they may have different sections and questions depending on whether the potential entrant 42 is being screened for the first time or has been previously screened, and depending on whether the potential entrant 42 has previously been cleared or not cleared for entry.

### D. Potential Entrant Records

Preferably, each potential entrant 42 that has requested entry to a facility 50 and that has been screened and evaluated for entry will have a corresponding potential entrant record created and maintained in the record storage of the entrant status and records component 26 of the electronic medical evaluation system 20. Preferably, the potential entrant records are searchable by some or all of the information and data contained in the records. This enables the electronic medical evaluation system 20 to provide centralized or distributed auditing, tracking, contact tracing, and reporting regarding a plurality of potential entrants 42 in a plurality of different locations, including their medical conditions, medical screening evaluations, facility entry decisions and status, etc. for a plurality of employers and proprietors 30 and a plurality of facilities 50 in a plurality of different locations.

As described above, employers and proprietors 30 preferably have real-time access to at least portions of the potential entrant records, excluding confidential information of potential entrants 42 that has been redacted or that is present only in authorized views 62. Also as described above, the electronic medical evaluation system 20 is adapted and configured to access and update the information in one or more records from time to time based on responses, information, and data provided by potential entrants 42 in response to questionnaires 46, information and data received from employers or proprietors 30, and/or information and data received from the review unit 60 and/or medical facilities 70, all as described herein. It will be appreciated that the ability to search, access and update the information in the potential entrant records preferably also includes the ability to display the records for viewing and selection by those authorized to do so.

Each potential entrant record may comprise a plurality of data fields. The data fields may correspond to and/or comprise information and data from potential applicant 42 responses to questionnaires 46, information and data automatically generated by the electronic medical evaluation system 20, information and data provided by employers and proprietors 30, and information and data provided from the review unit 60 and/or medical facilities 70.

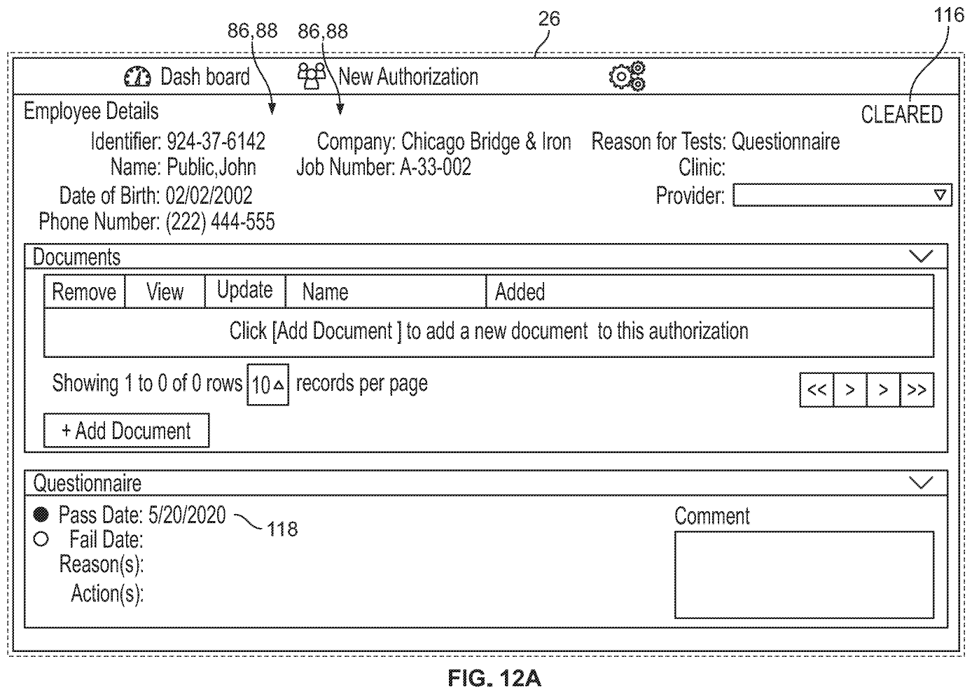

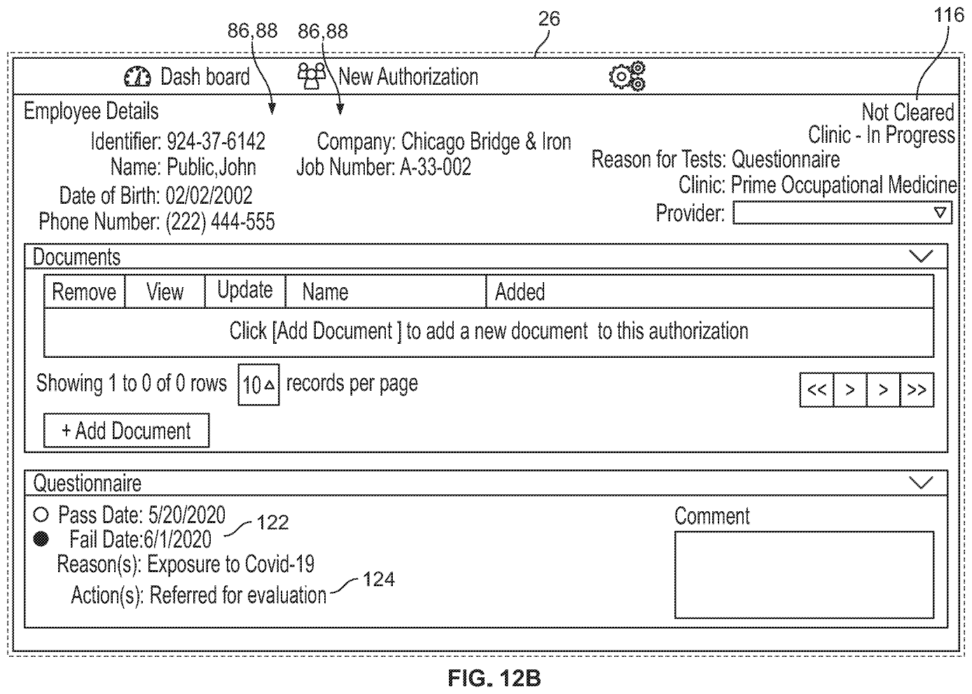

Example potential entrant records maintained in the record storage of the entrant status and records component 26 are illustrated in FIGS. 11 and 12A-12B. FIG. 11 illustrates an example summary list view of a plurality of example potential entrant records with example data fields displayed. The summary list view is adapted to provide a quick review of the identity and status of a plurality of potential entrants 42. The data fields displayed in the summary list view may include, for example entrant identification fields 86 such as the name and date of birth information of the potential entrant 42. The displayed data fields may also include employee identification fields 88 such as job identification (“job number”) and facility or location. The displayed data fields may also include entry status 116 as automatically generated by the electronic medical evaluation system 20, i.e., “cleared” or “not cleared.” The displayed data fields may also include the status date 118 of the potential entrants 42. The status date 118 may comprise the date the electronic medical evaluation system 20 automatically evaluated a potential entrant 42 and decided the potential entrant's entry status 116. It may also comprise a later date reflecting that the “cleared” or “not cleared” entry status of the potential entrant 42 subsequently changed due to subsequent exposure to an infectious disease, subsequent successful medical treatment, or otherwise. The entry status 116 and status dates 118 may thus reflect a history of the entry status of the potential entrant 42.

It will be appreciated that not all of the data fields comprising a potential entrant record need necessarily be displayed in the summary list view, and preferably will not be. However, it will be appreciated that fewer, more, or different data fields than those described above as examples may be displayed depending on individual preferences and needs.

Preferably, the potential entrant records shown in the summary list view of FIG. 11 are individually selectable to access a detail view that displays additional data fields of the selected record. FIGS. 12A and 12B illustrate example detail views of an example potential entrant record. The example detail view may comprise the same entrant identification and employee identification fields 86, 88 as the summary list view, as well as additional identification fields, for example employer name, potential entrant telephone number, and potential entrant identifier number. The example detail view may also comprise the same entry status 116 and status date 118 data fields as the summary list view, but may also display additional information regarding the potential entrant's entry status and entry status history. For example, as illustrated in FIGS. 12A and 12B, not only is the potential entrant's current “cleared” or “not cleared” entry status and date displayed, but also data fields for prior entry decision dates, the entry decisions made on those dates, i.e., cleared or not cleared, and data fields for “reason(s)” 122 and subsequent “actions” 124 related to the entry decisions. In FIG. 12B, for example, the additional data fields displayed show that the potential entrant 42 had an entry decision of cleared by the electronic medical evaluation system 20 on an earlier date, but an entry decision of not cleared on a subsequent date. The reason(s) 122 and actions(s) 124 data fields additionally show that the potential entrant 42 had a not cleared entry decision on the subsequent date due to exposure to Covid-19, and that the potential entrant 42 was referred for medical treatment. Also as seen in FIG. 12B, the entry status data field 116 includes the additional information that in addition to the potential entrant's current status as not cleared, the potential entrant 42 is currently undergoing medical treatment, i.e., “Clinic—In Progress.”

Similar to the summary list view described above, it will be appreciated that not all of the data fields comprising a potential entrant record need necessarily be displayed in the detail view, however they typically will be (except as precluded by privacy laws). Further, it will be appreciated that fewer, more, or different data fields than those described above as examples may be displayed, and that the arrangement of the data fields and the data they contain may differ depending on individual preferences and needs.

### E. Potential Entrant Interface

The potential entrant interface 40 of the medical screening system and method 10 comprises an interface that is adapted and configured for use by a potential entrant 42 in connection with requesting entry to a facility 50. The potential entrant interface 40 is adapted and configured for the potential entrant 42 to provide information and data regarding the potential entrant's identity and medical condition to the electronic medical evaluation system 20 for screening and evaluation before the potential entrant 42 can enter the facility 50.

More particularly, the potential entrant interface 40 is adapted and configured to send a request to the electronic medical evaluation system 20, and to receive an appropriate questionnaire 46 that elicits information concerning the potential entrant's 42 identity and medical condition. The potential entrant interface 40 is also adapted and configured to display the questionnaire 46 to elicit from the potential entrant 42 responses, information, and data in response to the questions comprising the displayed questionnaire 46, and to communicate the responses, information, and data to the electronic medical evaluation system 20 for processing, evaluation and generation of an entry decision. The potential entrant interface 46 is further adapted and configured to receive from the electronic medical evaluation system 20 an indication of the entry decision for the potential entrant 42 and facility 50, and in response to provide an entry decision presentation 48 corresponding to and reflecting the entry decision to enable entry control 54 to control entry to the facility 50 by the potential entrant 42. The potential entrant 42 may be any person desiring to enter a facility 50, including for example an employee, independent contractor, vendor, supplier, worker, customer, client, fan, visitor, attendee, or any other person. The potential entrant 42 may seek to enter the facility 50 for any reason, including for example to work, deliver supplies, attend an event, visit, dine, attend a meeting, or any other reason.

The potential entrant interface 40 is preferably adapted to be displayed or presented in, on, or by an electronic device. The potential entrant interface 40 may comprise an application that is executable on the electronic device. The application may be and preferably is downloadable to the electronic device, for example from an on-line app store, such as the Apple® or Google® on-line app store. The electronic device may comprise any of the electronic devices described herein, but in one embodiment preferably comprises a portable electronic device 80, such as any of those described herein. In another embodiment, the electronic device may comprise an electronic device that is permanently or temporarily located or fixed at or near an entrance to the facility 50. For example, such a device could include a display screen and be fixedly or removably mounted to a facility 50 to which entry is requested at or near an entrance.

The potential entrant interface 40 may be embedded within or hosted by the same electronic device as one or more components of the electronic medical evaluation system 20. For example, the potential entrant interface 40 and components of the electronic evaluation system 20 may be embedded in or hosted together by the portable electronic device 80 or another electronic device. The potential entrant interface 40 also may be embedded within or hosted by an electronic device that is separate from and perhaps remote to an electronic device on which one or more components of the electronic medical evaluation system 20 are embedded or hosted. For example, the potential entrant interface 40 may be embedded in or hosted by the portable electronic device 80 or another electronic device at a facility 50 to which entry is requested, and components of the electronic medical evaluation system 20 may be embedded in or hosted by a separate and perhaps remote electronic device, such as a remote computer or server.

The potential entrant interface 40 may also comprise a code reader and may be adapted and configured to receive and interpret or decode a code 52 encoding a selection of a particular questionnaire 46 or one or more factors to be used by the questionnaire storage/selection component 22 of the electronic medical evaluation system 20 to select a questionnaire 46. The code 52 may comprise a QR or bar code, for example, and may be provided at or by the facility 50 to which entry is requested. For example, the code 52 could be handed out at the facility 50 on a piece of paper or the like by an employer or proprietor 30. Alternatively, the code 52 could be physically posted on the facility 50 at or near an entrance. The potential entrant interface 40 may be adapted and configured to send the code 52 or information or data interpreted or decoded from the code 52 to the electronic medical evaluation system 20 with a request for a questionnaire 46. In an alternative embodiment, the code 52 could encode a questionnaire 46 so that a request to the electronic medical evaluation system 20 is not necessary.

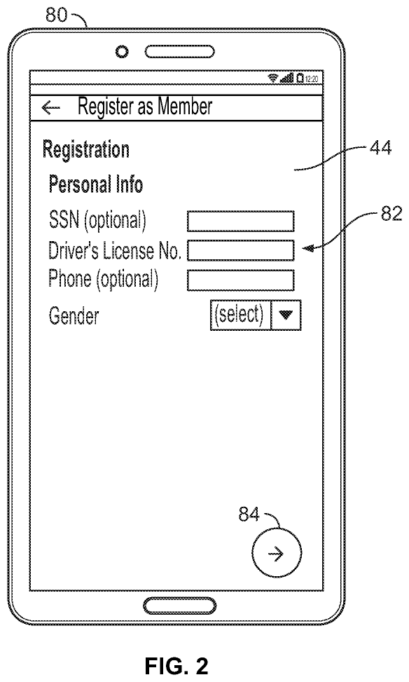

The potential entrant interface 40 may comprise a displayable registration/log-in page 44, such as illustrated in FIG. 2. The registration/log-in page 44 is preferably adapted and configured to enable a potential entrant 42 to register with the electronic medical evaluation system 20 and thereafter to log-in. The registration/log-in page 44 is preferably the first aspect of the potential entrant interface 40 that displays when the application comprising the potential entrant interface 40 executes. The registration/log-in page 44 preferably comprises one or more data entry fields 92 for a potential entrant 42 to enter information to register with the electronic medical evaluation system 20. The data entry fields 92 are preferably adapted to elicit information that uniquely identifies the potential entrant 42, for example social security number, driver's license number, telephone number, gender, and/or other information. The registration/login page 44 may also comprise other data entry fields for a user ID and a password.

The registration/log-in page 44 may also comprise a transmit selector 84, which may comprise a selectable button, icon, or the like for example. The transmit selector 84 is preferably selectable by the potential entrant 42 to cause the potential entrant interface 40 to send the information entered by the potential entrant 42 to the electronic medical evaluation system 20.

The registration/login page 44 may comprise a single page or multiple pages. For example, the registration/login page 44 may comprise a first displayable page comprising data entry fields for some or all of the information described above the first time a potential entrant 42 executes the application and registers with the electronic medical evaluation system 20, and a second different displayable page with only user ID and password fields after that.

The potential entrant interface 40 is preferably adapted and configured to send the request for a questionnaire 46 to the electronic medical evaluation system 20 upon successful registration and log-in, to receive the selected questionnaire 46 from the electronic medical evaluation system 20, and to display the received questionnaire 46 to elicit the potential entrant's responses, information, and data.

It will be appreciated that it may not be necessary for every potential entrant 42 to completely respond anew to a questionnaire 46 every time entry is requested to a facility 50. For example, after a potential entrant 42 has once registered, logged-in, and provided and submitted responses to a questionnaire 46, and the electronics medical evaluation system 20 has processed and stored the responses, information, and data, the electronic medical evaluation system 20 may automatically pre-populate the questionnaire 46 sent to the potential entrant interface 40 with some or all of the previously received, processed, and stored responses, information, and data the next time the potential entrant 42 logs in. In that event, the potential entrant 42 may only need to use the potential entrant interface 40 to review the pre-populated information, verify that it is still accurate and has not changed, or make the appropriate changes. Alternatively, upon log-in, the electronic medical evaluation system 20 and potential entrant interface 40 may be configured and adapted to simply inquire whether any of the potential entrant's information has changed and leave it at that. This may reduce the entry time for potential entrants 42 that have been previously cleared to enter facilities 50 and thus improve entry throughput especially in high volume situations such as large events at arenas, stadiums, or the like.

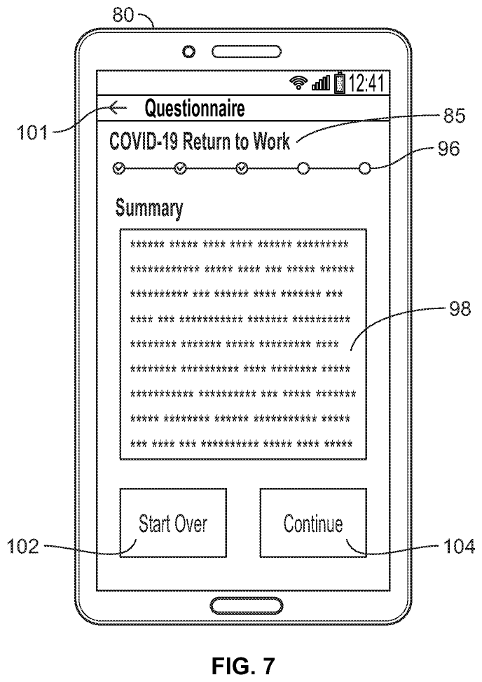

The potential entrant interface 40 may also comprise a displayable questionnaire summary page, such as illustrated in FIG. 7. The questionnaire summary page is preferably displayed when the potential entrant 42 selects the submit selector 93 of the questionnaire 46. The questionnaire summary page is preferably adapted and configured to enable a potential entrant 42 to review the responses, information, and data the potential entrant 42 provided to the questionnaire 46, correct or change the responses, information and/or data, and continue to the next steps in the process of completing and submitting the responses, information, and data to the electronic medical evaluation system 20 for processing.

The questionnaire summary page may comprise, for example, the questionnaire identifier 85, a progress indicator 96, and a summary 98. The progress indicator is adapted and configured to provide a visual indication of the potential entrant's progress in completing the process of providing and submitting questionnaire responses to the electronic medical evaluation system 20. The summary 98 is adapted and configured to display some or all of the responses, information, and/or data provided to the questionnaire 46 to enable the potential entrant 42 to review the responses, information, and/or data and make corrections or changes before they are sent to the electronic medical evaluation system 20 for processing.

The questionnaire summary page may also comprise, for example, a return to questionnaire selector 101, a start over selector 102 and/or a continue selector 104, each of which may comprise a selectable button, icon, or other visual element for example. The return to questionnaire selector 101 is preferably adapted and configured to cause the potential entrant interface 40 to return to displaying the questionnaire 46 to enable the potential entrant 42 to selectively add to, delete from, or otherwise change the responses, information and data previously provided. The start over selector 102 is preferably adapted and configured to cause the potential entrant interface 40 to clear the responses, information, and data previously provided and to return to displaying the questionnaire 46 to enable the potential entrant 42 to start the process of providing responses, information, and data over again. The continue selector 104 is preferably adapted and configured to indicate to the potential entrant interface 40 that the potential entrant 42 is satisfied with the responses, information, and data provided and to cause the potential entrant interface 40 to continue to the next step in the process of completing and sending the responses, information, and data provided to the electronic medical evaluation system 20.

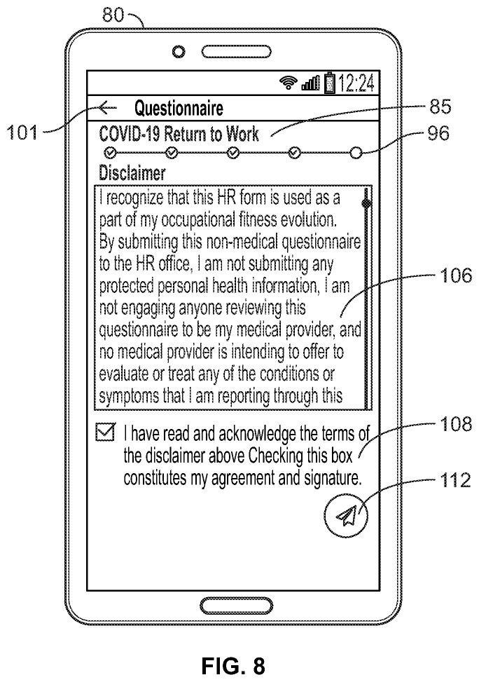

The potential entrant interface 40 may also comprise a displayable questionnaire disclaimer page, such as illustrated in FIG. 8. The questionnaire disclaimer page is preferably displayed when the potential entrant 42 selects the continue selector 104 of the questionnaire summary page. The questionnaire disclaimer page is preferably adapted and configured to provide to the potential entrant 42 any terms or conditions, legal or other notices or warnings, or any other information of which the potential entrant 42 should be aware, or is required to disclaim or acknowledge prior to the questionnaire responses, information and/or data being sent to the electronic medical evaluation system 20 for processing and evaluation.

The questionnaire disclaimer page may comprise, for example, the questionnaire identifier 85, the progress indicator 96, and the return to questionnaire selector 101. The questionnaire disclaimer page also preferably comprises a displayed disclaimer 106. The disclaimer 106 may include, for example, various terms, conditions, legal or other notices, warnings, acknowledgements, disclaimers, and the like which the potential entrant 42 is required to acknowledge and/or agree to as a condition of submitting the questionnaire responses, information, and data, and/or gaining access to a facility 50.

The questionnaire disclaimer page preferably also comprises an acknowledgement 108. The acknowledgement 108 preferably includes a statement of acknowledgement or agreement to the disclaimer 106 by the potential entrant 42, and may also comprise a statement, either in addition to or in place of a statement in the questionnaire 46, that the potential entrant 42 confirms and verifies that all responses, information, and data being provided are true and accurate to the best of the potential entrant's knowledge. The acknowledgement 108 may comprise a check box, icon, or other visual element that is selectable by the potential entrant 42 and is configured and adapted when selected to indicate to the potential entrant interface 40 that the potential entrant 42 acknowledges and agrees to the disclaimer 106 and, if included, verifies the information provided is true and correct. In addition to or instead of a selectable check box or other selectable visual element, the acknowledgement 108 may include one or more data entry fields for the potential entrant 42 to provide a signature or initials and a date.

The questionnaire disclaimer page also preferably comprises a submit selector 112 that may comprise a selectable button, icon, or other selectable visual element. The submit selector 112 is preferably adapted and configured when selected to indicate to the potential entrant interface 40 that the potential entrant 42 has agreed to the terms and conditions of the disclaimer 106, and to cause the potential entrant interface 40 to proceed to the next steps in the process of completing and submitting the questionnaire responses, information, and data to the electronic medical evaluation system 20. Preferably, the submit selector 112 is adapted and configured so that the potential entrant interface 40 will not proceed to the next steps in the process unless and until the potential entrant 42 has indicated agreement to the terms and conditions of the disclaimer 106 by selecting the acknowledgement 108.

The potential entrant interface 40 is preferably adapted and configured to display a submit questionnaire selector 114 in response to the potential entrant 42 selecting the submit selector 112 on the questionnaire disclaimer page. The submit questionnaire selector 114 is preferably adapted and configured to cause the potential entrant interface 40 to send the responses, information, and data provided by the potential entrant 42 in response to the questionnaire 46 to the electronic medical evaluation system 20 for processing, evaluation, and generation of an entry decision for the potential entrant 42.

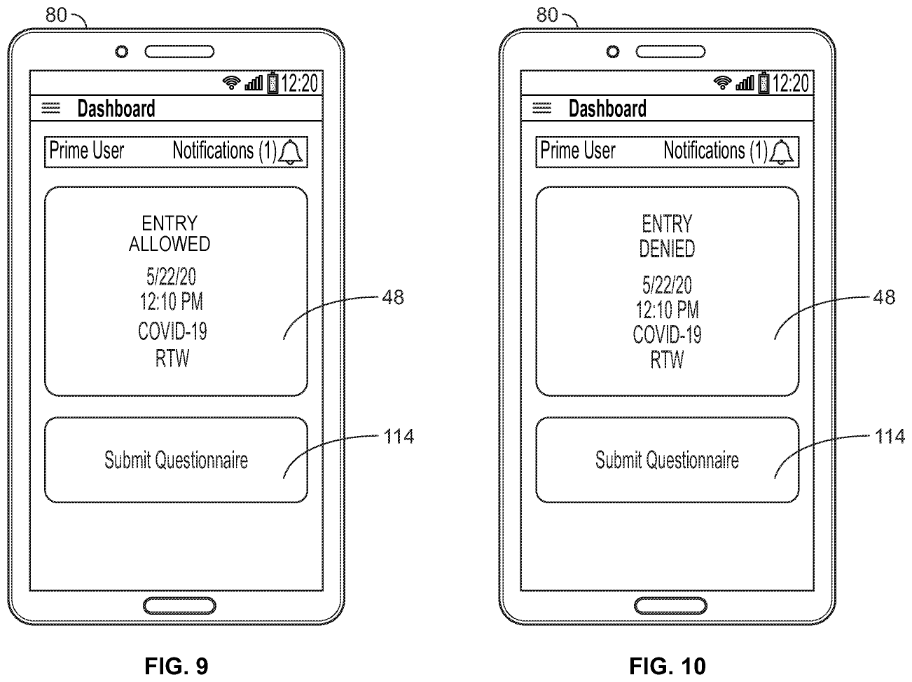

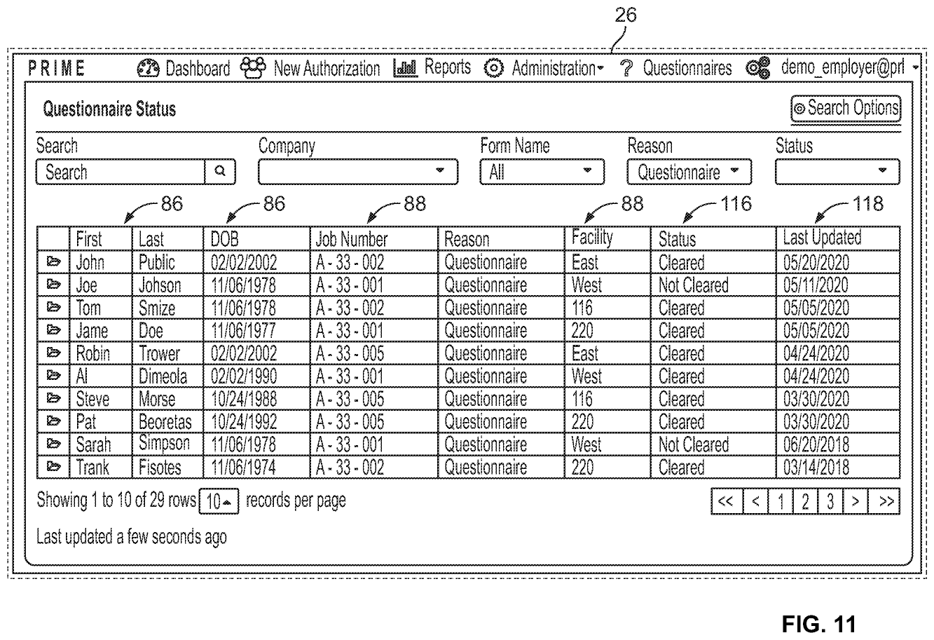

The potential entrant interface 40 preferably comprises an entry decision presentation page, such as illustrated in FIGS. 9-10. The potential entrant interface 40 is preferably adapted and configured to display the entry decision presentation page in response to receiving an entry decision indication from the electronic medical evaluation system 20 based on the evaluation of the questionnaire responses provided by the potential entrant 42. The entry decision presentation page preferably comprises an entry decision presentation 48. The potential entrant interface 40 is adapted and configured to provide the entry decision presentation 48 in response to the entry decision indication received from the electronic medical evaluation system 20 and in a form that corresponds to and reflects the entry decision.

The entry decision presentation 48 preferably comprises one or more visually perceptible elements corresponding to and reflecting the entry decision. For example, as illustrated in FIG. 9, the entry decision presentation 48 may comprise a displayed box of a first color, e.g. green, corresponding to and reflecting an entry decision of cleared, i.e., the potential entrant 42 is permitted entry to the facility 50. In contrast, as illustrated in FIG. 10, the entry decision presentation 48 may comprise a displayed box of a second color, e.g., red, corresponding to and reflecting an entry decision of not cleared, i.e., the potential entrant 42 is denied entry to the facility 50. The entry decision presentation 48 may also comprise other visually perceptible elements including, for example, other colors, shapes, blinking, scrolling, etc. For example, the entry decision presentation 48 may comprise a box or other shape of a third color, e.g., yellow, corresponding to a decision to delay an entry decision for a potential entrant 42.

The potential entrant interface 40 may also be adapted and configured to provide an entry decision presentation 48 that includes one or more non-visually perceptible auditory and/or tactile elements, such as sound and/or vibrations. An entry decision presentation 48 may comprise any number and combination of visually and non-visually perceptible elements consistent with indicating the nature of the entry decision and differentiating between different entry decisions. It will also be appreciated that the potential entrant interface 40 may be adapted and configured to provide an entry decision presentation 48 comprising a machine readable code corresponding to the entry decision. The machine readable code may be readable for example by a reader located at or near an entrance to the facility 50 and the reader may be adapted to automatically respond to a code corresponding to an entry decision of cleared to unlock and/or open the entrance.

The entry decision presentation 48 may also comprise other information related to the entry decision as illustrated in FIGS. 9-10. For example, the entry decision presentation 48 may comprise descriptive text, such as the date and time of the entry decision, a description of or related to the entry decision, e.g., “Entry Allowed” or “Entry Denied,” and/or a questionnaire identifier 85, e.g. “COVID-19 RTW.”

### F. Employers/Proprietors

The employer or proprietor 30 may be any company, any organization, regardless of legal form, any person, or any other entity regardless of legal form. The employer or proprietor 30 may comprise an employer as described in U.S. Patent Application Pub. Nos. US 2016/01804843 A1 and US 2016/0180292 A1, both previously incorporated herein by reference, and may perform the functions of an employer as described therein in addition to the functions described herein. An employer 30 may also be the proprietor 30 of a facility 50, or may be a separate entity. Further, multiple employers 30 may inhabit a single facility 30, and multiple proprietors 30 may control multiple sub-facilities within a facility 50. Any person or entity that controls entry by potential entrants 42 to a facility 50 or sub-facility within a facility 50 is considered an employer or proprietor 30 for purposes of the description herein without regard to the appropriate designation.

Employers and proprietors 30 may specify and provide the electronic medical evaluation system 20 with one or more potential entrant types 32 for which questionnaires 46 are required and entry to facilities 50 controlled. As described herein, potential entrant types may include, for example, employees, customers, vendors, suppliers, guests, visitors, attendees, and others.

Employers and proprietors 30 also may specify and provide the electronic medical evaluation system 20 with entry requirements 34 to be reflected in questionnaires 46 and to be used in making entry determinations. For example, entry requirements 34 may comprise various medical considerations, such as freedom from symptoms of specified infectious diseases. Further, the entry requirements 34 may vary depending on the entrant type as described herein.

Employers and proprietors 30 may be involved in questionnaire preparation and selection 35. As described herein, employers and proprietors 30 may prepare questionnaires 46 and provide them to the electronic medical evaluation system 20. Employers and proprietors 30 may also modify standard questionnaires 46 initially prepared by the entity in control of the electronic medical evaluation system 20 and provide them to the electronic medical evaluation system 20. Employers and proprietors may also specify factors to be used in selecting questionnaires 46 for potential entrants 42 and provide them to the electronic medical evaluation system 20.

Employers and proprietors 30 may also generate and provide to the electronic medical evaluation system 20 requests for medical evaluation 36 for potential entrants 42 as described herein. Employers and proprietors 30 may also specify and provide to the electronic medical evaluation system 20 standards 38 for the provision of medical evaluation to potential entrants 42 also as described herein.

### G. Facilities

A facility 50 may comprise any building or structure, or collection or grouping of buildings and/or structures, whether permanent or temporary, and whether enclosed, open, or partially open, including common carriers and other private public properties, which potential entrants 42 may request to enter and to which it is desired to control entry. A facility 50 may comprise a single premise or multiple premises. Preferably, a facility 50 will comprise one or more entrances at which entry control 54 may be exercised by manually and/or automatically managing and controlling entry by potential entrants 42. A facility 50 may comprise one or more codes 52 that may be readable by potential entrant interface 40 and that may specify one or more questionnaires 46 or factors for selecting questionnaires 46 for potential entrants 42 requesting entry to the facility 50 as described herein.

### H. Review Unit

The review unit 60 of the medical screening system and method 10 may comprise the reviewer described in U.S. Patent Application Pub. Nos. US 2016/01804843 A1 and US 2016/0180292 A1, both previously incorporated herein by reference, and may perform the functions of the reviewer as described therein in addition to the functions described herein. The review unit 60 may comprise any person, entity, or machine capable of reviewing the responses, information, and data provided to the review unit 60 and making the determinations from the same as described herein. The review unit 60 may comprise one or more doctors, nurses, medical technicians, clerical personnel, or others.

The review unit 60 is preferably adapted and configured to receive from the electronic medical evaluation system 20 responses, information, and data provided by potential entrants 42 in response to questionnaires 46. The review unit 60 is preferably adapted and configured to review and evaluate the same, redact confidential information, e.g., due to privacy laws, and/or generate authorized views 62 including such confidential information, and provide the information as redacted and/or the authorized views 62 to the electronic medical evaluation system 20. The review unit 60 may also be adapted and configured to generate, based on evaluation of the responses, information, and data, and to send to the electronic medical evaluation system 20 recommendations or proposals for medical evaluation for potential entrants 42.

The review unit 60 is also preferably adapted and configured to receive from the electronic medical evaluation system 20 information and data regarding medical evaluation, treatment and/or tests 72 and results 74 provided by a medical facility 70. The review unit 60 is preferably adapted and configured to review and evaluate the same, redact confidential information, e.g., due to privacy laws, and/or generate authorized views 62 including such confidential information, and provide the information as redacted and/or the authorized views 62 to the electronic medical evaluation system 20.

### I. Medical Facilities

A medical facility 70 may comprise any hospital, doctor's office, clinic, or other healthcare facility, and may further comprise one or more doctors, nurses, nurse practitioners, or other medical personnel authorized or licensed to perform requested medical evaluation, treatments and/or tests 72 on potential entrants 42. The medical facility 70 may comprise the medical clinic described in U.S. Patent Application Pub. Nos. US 2016/01804843 A1 and US 2016/0180292 A1, both previously incorporated herein by reference, and may perform the functions of the medical clinic as described therein in addition to the functions described herein.

The medical facility 70 is preferably adapted and configured to receive requests from the electronic medical evaluation system 20 to perform medical evaluation 72 on potential entrants 42 as described herein. The medical facility 70 is further adapted and configured to perform the requested medical evaluation 72 and to generate and provide the electronic medical evaluation system 20 with results 74. The medical facility 70 is preferably further adapted and configured to identify confidential information 76 therein and to notify the electronic medical evaluation system accordingly.

### J. Exemplary Telecommunications Networks

The medical screening system and method 10 may be utilized upon any telecommunications network capable of transmitting data including voice data and other types of electronic data. Examples of suitable telecommunications networks for the medical screening system and method include but are not limited to global computer networks (e.g. Internet), wireless networks, cellular networks, satellite communications networks, cable communication networks (via a cable modem), microwave communications network, local area networks (LAN), wide area networks (WAN), campus area networks (CAN), metropolitan-area networks (MAN), and home area networks (HAN). The medical screening system and method 10 may communicate via a single telecommunications network or multiple telecommunications networks concurrently. Various protocols may be utilized by the electronic devices for communications such as but not limited to HTTP, SMTP, FTP and WAP (wireless Application Protocol). The medical screening system and method 10 may be implemented upon various wireless networks such as but not limited to 3G, 4G, LTE, CDPD, CDMA, GSM, PDC, PHS, TDMA, FLEX, REFLEX, IDEN, TETRA, DECT, DATATAC, and MOBITEX. The medical screening system and method 10 may also be utilized with online services and internet service providers.

The Internet is an exemplary telecommunications network for the medical screening system and method. The Internet is comprised of a global computer network having a plurality of computer systems around the world that are in communication with one another. Via the Internet, the computer systems are able to transmit various types of data between one another. The communications between the computer systems may be accomplished via various methods such as but not limited to wireless, Ethernet, cable, direct connection, telephone lines, and satellite.

### K. Exemplary Central Communication Units

The medical screening system and method 10 may be utilized with one or more central communication units. For example, one or more components of the electronic medical evaluation system 20 may be embedded in or hosted by a central communication unit.

The central communication units may be comprised of one or more central communication sites with which communications may be established over the telecommunications network. The central communication units may be comprised of a server computer, cloud based computer, virtual computer, personal computer or other computer system or server capable of receiving and transmitting data via IP or other network protocols over the telecommunications network. As can be appreciated, a modem or other communication device may be required between each of the central communication units and the corresponding telecommunication networks. The central communications units may be comprised of any electronic system capable of receiving and transmitting information (e.g. voice data, computer data, etc.).

### L. Exemplary Mobile Devices

The medical screening system and method 10 may be utilized with one or more mobile devices. For example, one or more instances of potential entrant interfaces 40 and one or more instances of components of the electronic medical evaluation system 20 may be embedded in or hosted by one or more mobile devices. A mobile device or other electronic device may be utilized by a proprietor 30 of a facility 50 to which entry has been requested, and another may be carried and used by a potential entrant 42 requesting entry to the facility.

The mobile devices may be comprised of any type of mobile or portable computer or data device for practicing the various aspects of the medical screening system and method 10. For example, the mobile devices can comprise a personal computer (e.g. APPLE® based computer, an IBM based computer, or compatible thereof) or tablet computer (e.g. IPAD®). The mobile devices may also be comprised of various other electronic devices capable of sending and receiving electronic data including but not limited to smartphones, mobile phones, telephones, personal digital assistants (PDAs), mobile electronic devices, handheld wireless devices, two-way radios, smart phones, communicators, video viewing units, television units, television receivers, cable television receivers, pagers, communication devices, and digital satellite receiver units.

### M. Exemplary Programmable Computers and Servers

The medical screening system and method 10 may be utilized with one or more programmable computer systems and/or servers. For example, one or more components of the electronic medical evaluation system 20 may comprise or be embedded in or hosted by one or more programmable computer systems and/or servers configured and programmed to practice the various aspects of the medical screening system and method 10 as described above. Similarly, the employer/proprietors 30, facilities 50, medical review 60, and medical facilities 70 may each comprise one or more programmable computer systems and/or servers configured and programmed to practice the various aspects of the medical screening system and method 10 as described above.

The electronic medical evaluation system 20 preferably comprises a centralized communications and data hub of the medical screening system and method 10. Accordingly, the programmable computer systems and/or servers comprising the electronic medical evaluation system 20 are preferably coupled with and communicate with the programmable computer systems and/or servers of the employer/proprietors 30, facilities 50, medical review 60, and medical facilities 70 via suitable wired or wireless direct or networked connections, including for example via the telecommunications networks, exemplary central communication units, and/or exemplary mobile device described above.

Each of the computer systems and/or servers may include a display screen (or monitor), a printer, a hard disk drive, a network interface, and/or a keyboard. Each computer system and/or server will typically include a microprocessor, a memory bus, random access memory (RAM), read only memory (ROM), a peripheral bus, and perhaps a keyboard controller.

The microprocessor may comprise a general-purpose digital processor that controls the operation of the computer system or server. The microprocessor can be a single-chip processor or can be implemented with multiple components. Using program instructions retrieved from memory, the microprocessor will control the reception and manipulations of input data and the output and display of data on output devices.

The memory bus is utilized by the microprocessor to access the RAM and the ROM. RAM is used by the microprocessor as a general storage area and as scratch-pad memory, and can also be used to store input data and processed data. ROM can be used to store instructions or program code executable by microprocessor as well as other data.

The peripheral bus is used to access the input, output and storage devices of the computer system or server. These devices may include a display screen, a printer device, a hard disk drive, a network interface, and others.

A keyboard controller may be used to receive input from a keyboard and to send decoded symbols for each pressed key to microprocessor over bus. A keyboard may be used by a user to input commands and other instructions to the computer system or server. Other types of user input devices can also be used. For example, pointing devices such as a computer mouse, a track ball, a stylus, or a tablet to manipulate a pointer on a display screen of the computer system.

The display screen is an output device that displays images of data provided by the microprocessor via the peripheral bus or provided by other components in the computer or server. The printer device when operating as a printer provides an image on a sheet of paper or a similar surface.

The hard disk drive provides mass storage for various types of data and information storage for the computer or server. Alternatively, mass storage can comprise and be provided by a solid state disk (SSD), solid-state memory, or another suitable data storage device. The hard disk drive or alternate mass storage device of the computer systems and/or servers comprising the electronic medical evaluation system 20 preferably provide storage for the questionnaire storage and selection component 22 and for the entrant status and records component 26 of the electronic medical evaluation system 20.

The microprocessor together with an operating system operate to execute computer code and to process, produce, and use data. The computer code and data may reside on RAM, ROM, hard disk drive, or other storage device, such as EPROM, SSD, etc. The computer code and data can also reside on a removable program medium and can be loaded or installed onto the computer system or server when needed. Removable program mediums include, for example, CD-ROM, PC-CARD, USB drives, magnetic disk and magnetic tape.

The network interface circuit is utilized to send and receive data over a network connected to other computer systems. An interface card or similar device and appropriate software executable by the microprocessor can be utilized to connect the computer system to an existing network and transfer data according to standard protocols.

### N. Operation of Preferred Embodiment

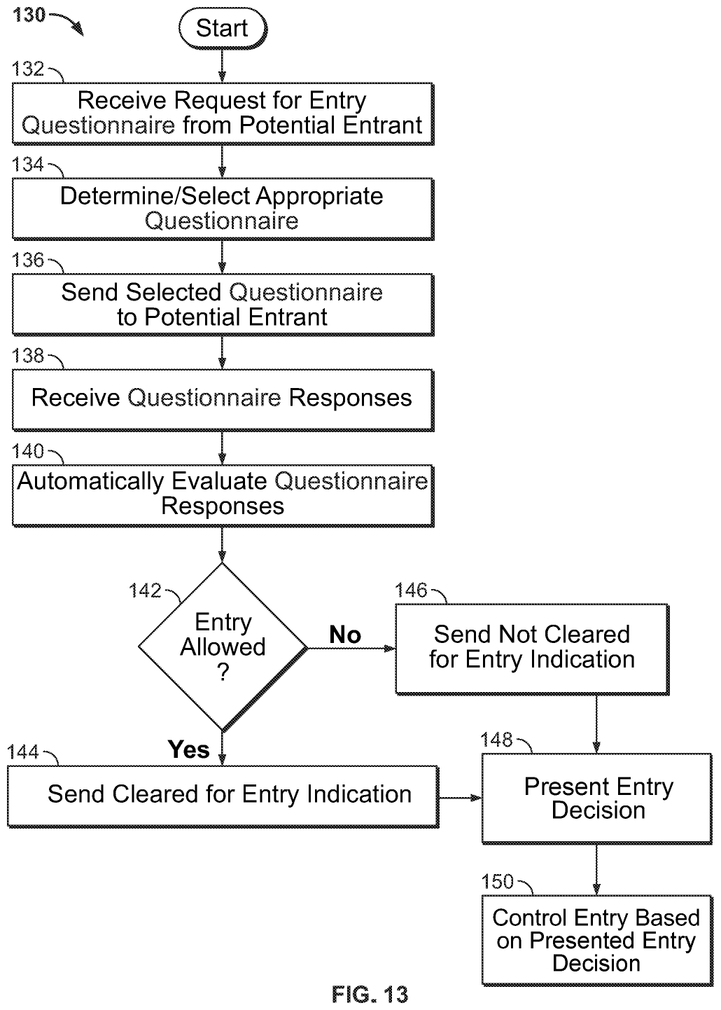

An example method 130 of using the example embodiments of the medical screening system and method 10 described above to medically screen and control entry of a potential entrant 42 to a facility 50 is described with reference to FIG. 13. In step 132 of the example method 130, the electronic medical evaluation system 20 receives a request for a questionnaire 46. As described herein, the request may be sent to the electronic medical evaluation system 20 by the potential entrant interface 40 in response to a potential entrant 42 logging-in via the potential entrant interface 40, or in response to the potential entrant interface 40 being used to read a code 52 at a facility 50 to which entry is requested.

In step 134, the electronic medical evaluation system 20 determines and selects an appropriate questionnaire 146. The questionnaire 146 may be selected based on one or more factors as described herein, including a type or identity of the potential entrant 42, a type or identity of the facility 50 to which entry is requested, a type or identity of a job of the potential entrant 42, etc.

In step 136, the electronic medical evaluation system 20 provides and sends the selected questionnaire 146 to the potential entrant interface 40. The potential entrant interface 40 receives and displays the questionnaire 46 and the questionnaire 46 elicits responses, information, and data from the potential entrant 42 regarding the potential entrant's identity and medical condition prior to the potential entrant 42 entering the facility 50.

In step 138, the electronic medical evaluation system 20 receives the responses, information, and data provided by the potential entrant in response to the questionnaire 46 via the potential entrant interface 40. In step 140, the electronic medical evaluation system 20 automatically processes and evaluates the responses, information, and data provided by the potential entrant 42, for example using one or more AI-based or other algorithms.

In determination step 142, the electronic medical evaluation system 20 automatically generates an entry decision, e.g. cleared or not cleared, based on the algorithmic evaluation of the responses, information, and data provided by the potential entrant 42. In alternate steps 144 and 146, the electronic medical evaluation system 20 sends alternate indications depending on the entry decision and information corresponding to the entry decision, i.e., cleared or not cleared, to the potential entrant interface 40.

In step 148, the potential entrant interface 40 provides an entry decision presentation 48 corresponding to and reflecting the entry decision. In step 150, entry to the facility 50 by the potential entrant 42 is controlled by the entry control 54 of the facility 50 in accordance with the entry decision presentation 48.

Any and all headings are for convenience only and have no limiting effect. Unless otherwise defined, all technical and scientific terms used herein have the same meaning as commonly understood by one of ordinary skill in the art to which this invention belongs. Although specific terms are employed herein, they are used in a generic and descriptive sense only and not for purposes of limitation. All publications, patent applications, patents, and other references mentioned herein are incorporated by reference in their entirety to the extent allowed by applicable law and regulations.

The data structures and code described in this detailed description are typically stored on a computer readable storage medium, which may be any device or medium that can store code and/or data for use by a computer system. This includes, but is not limited to, magnetic and optical storage devices such as disk drives, magnetic tape, CDs (compact discs), DVDs (digital video discs), and computer instruction signals embodied in a transmission medium (with or without a carrier wave upon which the signals are modulated). For example, the transmission medium may include a telecommunications network, such as the Internet.

At least one embodiment of the medical screening system and method is described above with reference to block and flow diagrams of systems, methods, apparatuses, and/or computer program products according to example embodiments of the invention. It will be understood that one or more blocks of the block diagrams and flow diagrams, and combinations of blocks in the block diagrams and flow diagrams, respectively, can be implemented by computer-executable program instructions. Likewise, some blocks of the block diagrams and flow diagrams may not necessarily need to be performed in the order presented, or may not necessarily need to be performed at all, according to some embodiments of the invention. These computer-executable program instructions may be loaded onto a general-purpose computer, a special-purpose computer, a processor, or other programmable data processing apparatus to produce a particular machine, such that the instructions that execute on the computer, processor, or other programmable data processing apparatus create means for implementing one or more functions specified in the flow diagram block or blocks. These computer program instructions may also be stored in a computer-readable memory that can direct a computer or other programmable data processing apparatus to function in a particular manner, such that the instructions stored in the computer-readable memory produce an article of manufacture including instruction means that implement one or more functions specified in the flow diagram block or blocks. As an example, embodiments of the invention may provide for a computer program product, comprising a computer usable medium having a computer-readable program code or program instructions embodied therein, the computer-readable program code adapted to be executed to implement one or more functions specified in the flow diagram block or blocks. The computer program instructions may also be loaded onto a computer or other programmable data processing apparatus to cause a series of operational elements or steps to be performed on the computer or other programmable apparatus to produce a computer-implemented process such that the instructions that execute on the computer or other programmable apparatus provide elements or steps for implementing the functions specified in the flow diagram block or blocks. Accordingly, blocks of the block diagrams and flow diagrams support combinations of means for performing the specified functions, combinations of elements or steps for performing the specified functions, and program instruction means for performing the specified functions. It will also be understood that each block of the block diagrams and flow diagrams, and combinations of blocks in the block diagrams and flow diagrams, can be implemented by special-purpose, hardware-based computer systems that perform the specified functions, elements or steps, or combinations of special-purpose hardware and computer instructions.

The present invention may be embodied in other specific forms without departing from the spirit or essential attributes thereof, and it is therefore desired that the present embodiment be considered in all respects as illustrative and not restrictive. Many modifications and other embodiments of the medical screening system and method will come to mind to one skilled in the art to which this invention pertains and having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although methods and materials similar to or equivalent to those described herein can be used in the practice or testing of the medical screening system and method, suitable methods and materials are described above. Thus, the medical screening system and method is not intended to be limited to the embodiments shown, but is to be accorded the widest scope consistent with the principles and features disclosed herein.

What is claimed is:

1. A system for medically screening a potential entrant to a facility, comprising:
an electronic medical evaluation system comprising a questionnaire, wherein the questionnaire comprises one or more questions concerning a medical condition or symptom of the potential entrant;
an interface configured to receive the questionnaire from the electronic medical evaluation system and to display the questionnaire;
wherein the electronic medical evaluation system is configured to receive responses to the one or more questions and to automatically generate an entry decision regarding entry to the facility by the potential entrant; and
wherein the electronic medical evaluation system is configured to automatically send an indication of the entry decision to the interface.

2. The system for medically screening a potential entrant to a facility of claim 1, wherein the electronic medical evaluation system comprises a plurality of questionnaires, and wherein the electronic medical evaluation system is configured to select the questionnaire from the plurality of questionnaires depending on a type or identity of the potential entrant.

3. The system for medically screening a potential entrant to a facility of claim 1, wherein the electronic medical evaluation system comprises a plurality of questionnaires, and wherein the electronic medical evaluation system is configured to select the questionnaire from the plurality of questionnaires depending on a type or identity of the facility.

4. The system for medically screening a potential entrant to a facility of claim 1, wherein the electronic medical evaluation system comprises a plurality of questionnaires, and wherein the electronic medical evaluation system is configured to select the questionnaire from the plurality of questionnaires depending on a type or identity of a job of the potential entrant.

5. The system for medically screening a potential entrant to a facility of claim 1, comprising a code, wherein the interface is configured to receive and interpret the code, and wherein the electronic medical evaluation system is configured to send the questionnaire to the interface in response to the code.

6. The system for medically screening a potential entrant to a facility of claim 5, wherein the interface comprises a portable data device, and wherein the code is provided at or near an entrance of the facility.

7. The system for medically screening a potential entrant to a facility of claim 5, wherein the electronic medical evaluation system comprises a plurality of questionnaires, and wherein the electronic medical evaluation system is configured to select the questionnaire from the plurality of questionnaires depending on the code.

8. The system for medically screening a potential entrant to a facility of claim 7, wherein the code comprises an indication of at least one of a facility type, facility identification, potential entrant type, potential entrant identification, and potential entrant job type.

9. The system for medically screening a potential entrant to a facility of claim 1, wherein the electronic medical evaluation system comprises a storage adapted to store a plurality of records corresponding to a plurality of potential entrants comprising one or more of a potential entrant identifier, entry decision, facility identifier, entry decision date, entry decision time, questionnaire identifier, and entry status history of the potential entrant.

10. The system for medically screening a potential entrant to a facility of claim 1, wherein the interface is adapted and configured to present a first perceptible element when the entry decision corresponds to the potential entrant being cleared for entry.

11. The system for medically screening a potential entrant to a facility of claim 10, wherein the first perceptible element comprises a first visually perceptible element.

12. The system for medically screening a potential entrant to a facility of claim 11, wherein the first visually perceptible element comprises a first color.

13. The system for medically screening a potential entrant to a facility of claim 10, wherein the first perceptible element comprises one of a first auditory perceptible element and a first tactile perceptible element.

14. The system for medically screening a potential entrant to a facility of claim 1, wherein the interface is adapted and configured to present a first perceptible element when the entry decision corresponds to the potential entrant being cleared for entry, and a second perceptible element different from the first perceptible element when the entry decision corresponds to the potential entrant being not cleared for entry.

15. The system for medically screening a potential entrant to a facility of claim 14, wherein the first perceptible element comprises a first visually perceptible element and the second perceptible element comprises a second visually perceptible element.

16. The system for medically screening a potential entrant to a facility of claim 15, wherein the first visually perceptible element comprises a first color, and the second visually perceptible element comprises a second color.

17. The system for medically screening a potential entrant to a facility of claim 14, wherein the first perceptible element comprises one of a first auditory perceptible element and a first tactile perceptible element, and the second perceptible element comprises one of a second auditory perceptible element and a second tactile perceptible element.

18. The system for medically screening a potential entrant to a facility of claim 14, wherein the interface is adapted and configured to present a third perceptible element different from the first perceptible element and the second perceptible element when the entry decision corresponds to the entry decision being delayed.

19. The system for medically screening a potential entrant to a facility of claim 1, wherein the indication of the entry decision comprises a machine readable code indicative of the entry decision.

20. The system for medically screening a potential entrant to a facility of claim 1, wherein the one or more questions relate to one or more of potential entrant identification, potential entrant type, potential entrant job type, potential entrant job identification, potential entrant travel locations, exposure to disease, symptoms, tests performed, test results, and diagnoses received.

21. The system for medically screening a potential entrant to a facility of claim 1, wherein the electronic medical evaluation system is configured to generate a request for medical evaluation for the potential entrant depending on the entry decision.

22. A method of using the system for medically screening a potential entrant to a facility of claim 1, comprising:
receiving at the electronic medical evaluation system a request from the interface for the questionnaire;
sending the questionnaire from the electronic medical evaluation system to the interface;
displaying the questionnaire at the interface;
receiving responses to the one or more questions at the electronic medical evaluation system and using the electronic medical evaluation system to automatically generate an entry decision regarding entry to the facility by the potential entrant based on the responses; and
automatically sending an indication of the entry decision from the electronic medical evaluation system to the interface.

23. The method of using the system for medically screening a potential entrant to a facility of claim 22, wherein using the electronic medical evaluation system to automatically generate an entry decision comprises applying a decision algorithm to the responses or reviewing the responses by a human reviewer.

24. The method of using the system for medically screening a potential entrant to a facility of claim 22, comprising selecting the questionnaire from a plurality of questionnaires depending on a type or identity of the potential entrant, type or identity of a job of the potential entrant, or a type or identity of the facility.

25. The method of using the system for medically screening a potential entrant to a facility of claim 22, comprising:
reading a code provided at or near an entrance of the facility with the interface; and
selecting the questionnaire from the plurality of questionnaires depending on the code.

26. The method of using the system for medically screening a potential entrant to a facility of claim 25, wherein the code comprises an indication of at least one of a facility type, facility identification, potential entrant type, potential entrant identification, and potential entrant job type.

27. The method of using the system for medically screening a potential entrant to a facility of claim 22, comprising presenting with the interface a first visual, auditory, or tactile perceptible element corresponding to the entry decision clearing the potential entrant for entry, and second visual, auditory, or tactile perceptible element corresponding to the entry decision not clearing the potential entrant for entry.

28. The method of using the system for medically screening a potential entrant to a facility of claim 27, comprising presenting with the interface a third visual, auditory, or tactile perceptible element corresponding to the entry decision being delayed.

29. The method of using the system for medically screening a potential entrant to a facility of claim 22, comprising the electronic medical evaluation system generating a request for medical evaluation for the potential entrant depending on the entry decision.
